(19) United States
(12) Patent Application Publication
(10) Pub. No.: US 2010/0196308 A1
(43) Pub. Date: Aug. 5, 2010

(54) VITAMIN D3 AND ANALOGS THEREOF FOR ALLEVIATING SIDE EFFECTS ASSOCIATED WITH CHEMOTHERAPY

(76) Inventors: Joaquin J. Jimenez, Miami, FL (US); John Patrick McCook, Frisco, TX (US); Niven Rajin Narain, Cambridge, MA (US)

Correspondence Address: MCCARTER & ENGLISH, LLP BOSTON, 265 Franklin Street, Boston, MA, 02110, US

(21) Appl. No.: 12/695,113

(22) Filed: Jan. 27, 2010

Related U.S. Application Data
(60) Provisional application No. 61/147,549, filed on Jan. 27, 2009.
(60) Provisional application No. 61/239,003, filed on Sep. 1, 2009.

(51) Int. Cl. A61K 38/19; A61K 38/22; A61P 7/06; A61K 31/593

(52) U.S. Cl. 424/85.1; 514/8; 514/167

(57) ABSTRACT

The present disclosure relates to the use of vitamin D compounds, such as vitamin D3, or analogs and/or metabolites thereof, to modulate bone marrow progenitors and stromal cells prior to the administration of antineoplastic agents. The methods of the present disclosure may ameliorate myelosuppression by increasing the availability of pluripotent stem cell progenitors, and can be used in combination with standard therapy (e.g. granulocyte stimulating factor) to increase proliferation of myeloid cells and/or improve their mobilization from the bone marrow, thereby diminishing the dose and administration of colony-stimulating factors (CSFs) as well as the recuperation time following chemotherapy.

### RELATED APPLICATIONS

This application claims priority to U.S. Provisional Patent Application No. 61/147,549, filed Jan. 27, 2009 and U.S. Provisional Patent Application No. 61/239,003, filed Sep. 1, 2009. The contents of each of the foregoing applications are hereby incorporated in their entirety.

### TECHNICAL FIELD

The present disclosure provides for the use of vitamin D compounds, such as vitamin D3 and analogs thereof, having calcemic and non-calcemic activity, administered in a pharmaceutically acceptable manner prior to the administration of anti-neoplastic drugs to treat solid tumors and/or leukemia.

### BACKGROUND OF THE INVENTION

Compositions for treating cancer are constantly being developed and tested. For example, vitamin D3 analogs have emerged in the field of cancer treatment as potent cell differentiators. One of the most widely used and studied, 1,25(OH)₂D3 (calcitriol), has been demonstrated to induce differentiation alone and in combination with colony stimulating factors in myelodysplastic disorders (MDS). In fact, a method to treat MDS with 1,25(OH)₂D3 by administering high pulse doses has been developed to avoid hypercalcemia, the most significant side effect of this analog.

One issue with cancer treatments is the side effects that accompany most available treatments. Specifically, cytotoxic chemotherapies are administered systemically to eliminate cancer cells due to their unusually high proliferative rate. Such regimes, however, cannot distinguish between normal cells in their proliferative stage and, therefore, all cells in the active growth phase will be targeted by chemotherapeutic agents. As a result, anti-neoplastic therapies unavoidably cause serious side effects, such as chemotherapy-induced myelosuppression (CIM), which induces anemia, thrombocytopenia and neutropenia, leading to fatigue, increased bleeding and an increased risk of serious infections.

Accordingly, it is desirable to provide methods for reducing and/or alleviating the side effects of chemotherapeutic agents suffered by subjects undergoing chemotherapeutic treatment.

### SUMMARY OF THE INVENTION

The present invention provides methods for protecting pluripotent stem cells and growth-factor producing stromal cells from secondary toxicity due to chemotherapy administration. In certain embodiments, vitamin D compounds, such as vitamin D3 and/or its analogs or metabolites, including, but not limited to calcitriol (1,25(OH)₂D3), may be used to modulate bone marrow progenitors and stromal cells prior to the administration of anti-neoplastic agents.

In certain embodiments, the vitamin D compounds of the invention (e.g., vitamin D3 and/or its analogs or metabolites) can be administered in a manner such that hypercalcemia or interference with anti-neoplastic treatments can be avoided.

In other embodiments, a patient's myeloid cells may be screened prior to the administration of the subject vitamin D compound (e.g., vitamin D3 and/or its analogs or metabolites thereof) to determine the optimal dose for protection, without eliciting a hypercalcemic effect.

In yet other embodiments, the invention provides methods of preventing or reducing chemotherapy-induced myelosuppression in a subject being treated with a chemotherapeutic agent which induces myelosuppression by administering to the subject an effective amount of a vitamin D compound or a pharmaceutically acceptable salt, prodrug or solvate thereof.

In other embodiments, the invention provides methods of preventing or reducing the risk of myelosuppression induced disorders in a subject being treated with a chemotherapeutic agent that induces myelosuppression by administering to the subject an effective amount of a vitamin D compound or a pharmaceutically acceptable salt, prodrug or solvate thereof.

In some embodiments, the invention provides methods of preventing depletion of neutrophils in a subject being treated with a chemotherapeutic agent by administering to the subject an effective amount of a vitamin D compound or a pharmaceutically acceptable salt, prodrug or solvate thereof.

### BRIEF DESCRIPTION OF THE DRAWINGS

Various embodiments of the present disclosure will be described herein below with reference to the figures wherein:

FIG. 1A is a photomicrograph of a colony of untreated stem cells that was utilized as a control.

FIG. 1B is a photomicrograph of a colony of stem cells treated only with 1,25(OH)₂D3.

FIG. 1C is a photomicrograph of a colony of stem cells treated with 1,25(OH)₂D3 in conjunction with 4-hydroxyperoxycylophosphamide (4-HC).

FIG. 2 is a graph measuring viability of myeloid cells by trypan blue exclusion after exposure to various doses of 1,25(OH)₂D3.

FIGS. 3(a)-(c) provides graphs comparing the absolute neutrophil counts of rats treated with a first cycle of (a) cyclophosphamide and vehicle (O) or cyclophosphamide and calcitriol (); (b) cyclophosphamide plus doxorubicin (O) and vehicle or cyclophosphamide plus doxorubicin and calcitriol (); and (c) cyclophosphamide, doxorubicin and paclitaxel and vehicle (O) or cyclophosphamide, doxorubicin and paclitaxel and calcitriol ().

FIGS. 4(a)-(c) provides charts comparing the number of colonies obtained from bone marrow cultures on day 22 during the first cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

FIGS. 5 (a)-(c) provides charts comparing the number of colonies obtained from bone marrow cultures on day 25 during the first cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

FIGS. 6(a)-(c) provides charts comparing the number of colonies obtained from bone marrow cultures on day 32 during the first cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

FIGS. 7(a)-(c) provides graphs comparing the absolute neutrophil counts of rats treated with a second cycle of (a) cyclophosphamide and vehicle (o) or cyclophosphamide and calcitriol (); (b) cyclophosphamide plus doxorubicin (o) and vehicle or cyclophosphamide plus doxorubicin and calcitriol (); and (c) cyclophosphamide, doxorubicin and paclitaxel and vehicle (o) or cyclophosphamide, doxorubicin and paclitaxeland calcitriol ().

FIGS. 8(a)-(c) provides charts comparing the number of colonies obtained from bone marrow cultures on day 49 during the second cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

FIGS. 9(a)-(c) provides charts comparing the number of colonies obtained from bone marrow cultures on day 52 during the second cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

FIGS. 10(a)-(c) provides charts comparing the number of colonies obtained from bone marrow cultures on day 60 during the second cycle of treatment of rats with (a) control, cyclophosphamide and vehicle or cyclophosphamide and calcitriol; (b) control, cyclophosphamide plus doxorubicin and vehicle or cyclophosphamide plus doxorubicin and calcitriol; and (c) control, cyclophosphamide, doxorubicin and paclitaxel and vehicle or cyclophosphamide, doxorubicin and paclitaxel and calcitriol.

### DETAILED DESCRIPTION OF THE INVENTION

Differentiated cells are not susceptible to chemotherapy for reasons that are incompletely elucidated. Therefore, maintaining a balance between the minimum amount of progenitors necessary to sustain life and the need to eradicate the malignant cells is often dependent on the patient's progenitor pool being able to withstand the toxic onslaught of chemotherapy, and then repopulate the bone marrow and allow the progenitors to be mobilized by different growth factors. Maintaining such a balance is a challenge most oncologists face, and has an impact on the therapeutic approach employed leading, for example, to decreased doses of chemotherapy, less cycles, and the use of adjuvant therapies which can have a negative impact on the survival outcome of a patient.

Perhaps the most radical example of this phenomenon is bone marrow ablation, a necessary treatment for some types of leukemia. Bone marrow ablation has alarmingly high mortality rates, mostly due to secondary effects of extreme CIM.

Thus, a regime that protects normal myeloproliferative cells would lead to significant decreases in both mortality and morbidity amongst patients with different forms of cancer. To this date, palliative approaches, such as modified chemotherapy protocols and the use of different hematopoietic factors are in favor. One of the main concerns for the use of a protective agent to modulate normal bone marrow cells is that it may interfere with antineoplastic agents and therefore decrease the chances of cancer remission. Thus, CIM is nowadays treated empirically by decreasing chemotherapy doses when white blood cell counts are critical, and by administrating growth factors such as G-CSF and erythropoietin (EPO) to counteract chemotherapy-induced anemia. For example, neutropenia (a decrease of the neutrophil granulocyte count below 0.5×10⁹/L) can be improved with synthetic G-CSF (granulocyte-colony stimulating factor, e.g., pegfilgrastim, filgrastim, lenograstim). This approach has led to shorter amelioration time. However, they may carry a significant burden of unpleasant side effects to patients such as fever, chills, extensive bone pain, which, when compounded with other side effects of antineoplastic therapy, lead to a decrease in quality of life, as well as an onerous social cost because of the high expense of recombinant colony stimulating factors.

Thus in one aspect, the invention provides methods of preventing or reducing chemotherapy-induced myelosuppression in a subject being treated with a chemotherapeutic agent which induces myelosuppression by administering to the subject an effective amount of a vitamin D compound or a pharmaceutically acceptable salt, prodrug or solvate thereof. The language “chemotherapy-induced myelosuppression (CIM)” includes a decrease in the number of blood cells (e.g., red blood cells, white blood cells, such as neutrophils and/or platelets) that occurs upon treatment of a subject with one or more chemotherapeutic agents that induces myelosuppression. In one embodiment, CIM causes anemia (e.g., due to the decrease in the number of red blood cells). Symptoms of anemia include, for example, weakness, fatigue, malaise, poor concentration, shortness of breath, heart palpitations, angina, pallor, tachycardia, and cardiac enlargement. In another embodiment, CIM causes neutropenia (e.g., due to the decrease in the number of neutrophils). Symptoms of neutropenia include, for example, an increase risk of severe infection or sepsis, fevers, mouth ulcers, diarrhea and sore throat. In yet another embodiment, CIM causes thrombocytopenia (e.g., due to the decrease in the number of platelets). Symptoms of thrombocytopenia include, for example, an increased risk of bleeding, purpura, nosebleeds and bleeding gums.

The language “preventing CIM” includes the arresting or suppression of CIM or one or more symptoms associated with CIM.

The language “reduction,” reduce” and “reducing” includes the diminishment, alleviation or complete amelioration of CIM or one or more symptoms associated with CIM.

The term “subject” includes mammals, e.g., cats, dogs, horses, pigs, cows, sheep, rodents (e.g., rats, mice), rabbits, squirrels, bears, primates (e.g., chimpanzees, gorillas, and humans) which are capable of suffering from CIM. In one embodiment, the subject is a rat. In other embodiments, the subject is a genetically modified mammal. In yet another embodiment, the subject is a human.

The language “chemotherapeutic agent” includes antineoplastic agents (e.g., chemical compounds that inhibit the growth of an abnormal tissue mass) used to treat cancer, antibiotics, or other cytostatic chemotherapeutic agents (e.g., that treat multiple sclerosis, dermatomyositis, polymyositis, lupus, rheumatoid arthritis and the suppression of transplant rejections). In one embodiment, the chemotherapeutic agent includes those agents that induce CIM. Examples of chemotherapeutic agents include, for example, alkylating agents (e.g., cisplatin, carboplatin, oxaliplatin, mechlorethamine, cyclophosphamide, chlorambucil or ifosfamide), antimetabolites (e.g., purine, for example, azathioprine, mercaptopurine, or pyrimidine), plant alkaloids (e.g., vinca alkaloids such as vincristine, vinblastine, vinorelbine and vindesine), taxanes (e.g., paclitaxel and docetaxel), podophyllotoxins (e.g., etoposide and teniposide), topoisomerase inhibitors (e.g., amsacrine) and anti-tumor antibiotics (e.g., dactinomycin, doxorubicin, epirubicin and bleomycin). In some embodiments, the chemotherapeutic agents include doxorubicin, paclitaxel and/or cyclophosphamide and any combinations thereof.

In one embodiment, the chemotherapeutic agent is a cell-cycle specific agent. The language “cell-cycle specific agent” includes chemotherapeutic agents that target a specific cycle of cell growth. In other embodiments, the chemotherapeutic agent is a nonspecific cell-cycle agent. The language “nonspecific cell-cycle agent” includes chemotherapeutic agents that target any or all cycles of cell growth. Examples of nonspecific cell-cycle agents include, for example, alkylating agents such as nitrogen mustards (e.g., cyclophosphamide, mechlorethamine, uramustine, melphalan, chlorambucil and ifosfamide) nitrosoureas (e.g., carmustine, lomustine and streptozocin) and alkyl sulfonates (e.g., busulfan); alkylating-like agents, such as cisplatin, carboplatin, nedaplatin, oxaplatin, satraplatin, and triplatin tetranitrate; or procrabazine and altretamine.

In some embodiments, the subject is being treated with a combination of chemotherapeutic agents (e.g., more than one chemotherapeutic agent). Accordingly, the combination of chemotherapeutic agents may include cell-cycle specific agents, cell-cycle non specific agents, or a combination thereof.

The language “treat with a chemotherapeutic agent” includes the administration to a subject of one or more of chemotherapeutic agents in a manner appropriate for treating the condition for which the chemotherapeutic agent is being administered (e.g., cancer).

In other embodiments, the invention provides methods of reducing the risk of or preventing myelosuppression induced disorders in a subject being treated with a chemotherapeutic agent that induces myelosuppression by administering to the subject an effective amount of a vitamin D compound or a pharmaceutically acceptable salt, prodrug or solvate thereof.

The language “myelosuppression-induced disorders” includes those disorders and symptoms of the disorders that occur as a result of chemotherapy-induced myelosuppression. Examples of myelosuppression-induced disorders includes myelosuppression-induced anemia (which include such symptoms as, for example, weakness, fatigue, malaise, poor concentration, shortness of breath, heart palpitations, angina, pallor, tachycardia, and cardiac enlargement), myelosuppression-induced neutropenia (which includes such symptoms as, for example, an increase risk of severe infection or sepsis, fevers, mouth ulcers, diarrhea and sore throat) or myelosuppression-induced thrombocytopenia (which include such symptoms as for example, an increased risk of bleeding, purpura, nosebleeds and bleeding gums).

In one embodiment, the myelosuppression-induced disorder is myelosuppression-induced neutropenia. In yet another embodiments, the myelosuppression-induced disorder is myelosuppression-induced infection, myelosuppression-induced fevers, myelosuppression-induced mouth ulcers, myelosuppression-induced diarrhea and myelosuppression-induced sore throat. The language “myelosuppression induced infection” includes infections (e.g., sepsis) that occur as a result of chemotherapy induced myelosuppression and/or chemotherapy induced neutropenia.

In some embodiments, the invention provides methods of preventing depletion of neutrophils in a subject being treated with a chemotherapeutic agent by administering to the subject an effective amount of a vitamin D compound or a pharmaceutically acceptable salt, prodrug or solvate thereof.

The language “preventing depletion of neutrophils” includes the arresting or suppression of the loss of neutrophils in a subject that can occur as a result of treating the subject with a chemotherapeutic agent. In some embodiments, the methods of the invention prevent the depletion of neutrophils by at least about 5%, about 10%, about 15%, about 20%, about 25%, about 30%, by about 35%, by about 40%, by about 45%, by about 50%, by about 55%, by about 60%, by about 65%, by about 70%, by about 75%, by about 80%, by about 85%, by about 90%, by about 95% or by about 100%.

The language “administer,” “administering” and “administration” includes providing one or more doses of the vitamin D compound in an amount effective to prevent or reduce CIM. Optimal administration rates for a given protocol of administration of the vitamin D compound can ascertained by those skilled in the art using conventional dosage determination tests conducted with regard to the specific compounds being utilized, the particular compositions formulated, the mode of application, the particular site of administration and the like.

In one embodiment, the vitamin D compound is administered in a pulsed dose. The language “pulsed dose” includes the administration of a dose of a vitamin D compound repetitively administered over a short period of time.

In some embodiments, the dose of vitamin D compound administered to the subject is between about 0.1 μg/m² and about 300 μg/m², between about 1 μg/m² and 280 μg/m², between about 25 μg/m² and about 260 μg/m². In other embodiments, the dose of the vitamin D compound administered to the subject is between about 10 μg/kg and about 200 μg/kg.

In one embodiment, the vitamin D compound is administered prior to administration of the chemotherapeutic agent. The vitamin D compound may be administered about 5 minutes, about 10 minutes, about 20 minutes, about 30 minutes, about 45 minutes, about an hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 36 hours, about 48 hours, about 60 hours, about 72 hours, about 84 hours or about 96 hours prior to the administration of the chemotherapeutic agent.

In other embodiments, the vitamin D compound is administered at substantially the same time as the chemotherapeutic agent. For example, the vitamin D compound may be co-administered with the chemotherapeutic agent; the vitamin D compound may be administered first, and immediately followed by the administration of the chemotherapeutic agent or the chemotherapeutic agent may be administered first, and immediately followed by the administration of the vitamin D compound.

In one embodiment, the vitamin D compound is administered after administration of the chemotherapeutic agent. The vitamin D compound may be administered about 5 minutes, about 10 minutes, about 20 minutes, about 30 minutes, about 45 minutes, about an hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours after the administration of the chemotherapeutic agent.

In some embodiments, administration of the vitamin D compound does not substantially increase calcium levels in the subject. In another embodiment, administration of the vitamin D compound does not induce hypercalcemia (e.g., too much calcium or abnormally high calcium in the blood).

In other embodiments, the vitamin D compound is co-administered with an additional agent that counteracts chemotherapy-induced toxicity, for example, bone marrow side effects such as chemotherapy-induced anemia. The language “chemotherapy-induced anemia” includes anemia (e.g., a decrease in the amount of red blood cells) that occurs as result of administration of a chemotherapeutic agent. The language “an agent that counteracts chemotherapy-induced anemia” includes those agents that treat, prevent, reduce or ameliorate chemotherapy-induced anemia or one or more symptoms thereof. In some embodiments, additional agent that counteracts chemotherapy-induced anemia includes growth factors, for example, epoetin alfa, erythropoietin (EPO) or granulocyte colony stimulating factor (G-CSF). For example, the agent may be a growth factor, such as G-CSF, GM-CSF, PDGF, EGF, or EPO.

The language “effective amount” of the compound is that amount necessary or sufficient to prevent or reduce CIM or one or more symptoms of CIM in a subject. The effective amount can vary depending on such factors as the size and weight of the subject, the type of illness, etc. One of ordinary skill in the art would be able to study the aforementioned factors and make the determination regarding the effective amount of the vitamin D compound without undue experimentation.

In one embodiment, the vitamin D compound is represented by Formula (I):

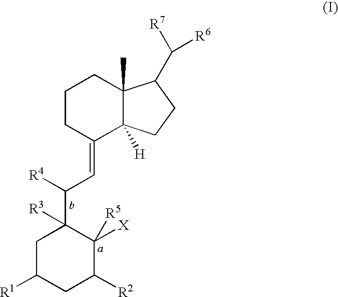

wherein

a and b are each independently a single or double bond

X is —CH₂ when a is a double bond, or X is hydrogen or a hydroxyl substituted alkyl when a is a single bond;

R¹ is hydrogen, hydroxyl, alkoxyl, tri-alkyl silyl or a substituted or unsubstituted alkyl, independently substituted with one to three halogen, hydroxyl, cyano or —NR′R″ moieties;

R² is hydrogen, hydroxyl, —O-trialkyl silyl, or a substituted or unsubstituted alkyl, alkoxyl or alkenyl, independently substituted with one to three halogen, hydroxyl, cyano or —NR′R″ moieties;

R³ is absent when b is a double bond or R³ is hydrogen, hydroxyl or alkyl, or R³ and R¹ together with the carbon atoms to which they are attached may be linked to form 5-7 membered carbocyclic ring when b is a single bond;

R⁴ is hydrogen, halogen or hydroxyl;

R⁵ is absent when a is a double bond or R⁵ is hydrogen, halogen or hydroxyl when a is a single bond;

R⁶ is a substituted or unsubstituted alkyl, alkenyl, alkynyl, cycloalkyl, heterocyclicyl, alkyl-O-alkyl, alkyl-CO₂-alkyl independently substituted with one to five, hydroxyl, oxo, halogen, alkoxyl, aryl, heteroaryl, cyano, nitro or —NR′R″ moieties;

R⁷ is a substituted or unsubstituted alkyl independently substituted with one to three hydroxyl, halogen, alkoxyl, aryl, heteroaryl, cyano, nitro or —NR′R″ moieties; and,

R′ and R″ are each, independently, hydrogen, hydroxyl, halogen, —C₁₋₇ alkyl or —C₁₋₇ alkoxyl.

In some embodiments, R¹ is hydroxyl, R² is hydroxyl, a is a double bond, R⁵ is absent, X is —CH₂, b is a double bond, R³ and R⁴ are absent, R⁶ is alkyl (e.g., methyl), R⁷ is alkyl (e.g., a substituted or unsubstituted C₅ alkyl, for example, a hydroxyl substituted C₅ alkyl or a cycloalkyl substituted C₅ alkyl).

In certain embodiments, the vitamin D compound is represented by Formula (II):

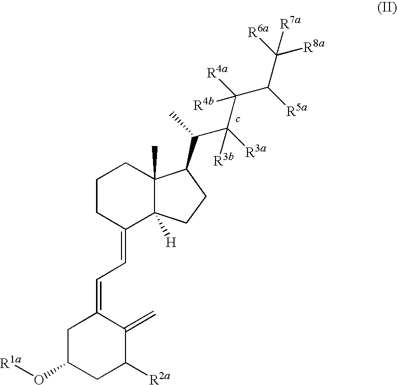

wherein

c is a single or double bond;

R^{1a} is hydrogen, tri-alkyl silyl or a substituted or unsubstituted alkyl, independently substituted with one to three halogen, hydroxyl, cyano or —NR′R″ moieties;

R^{2a} is hydrogen, hydroxyl, —O-trialkyl silyl, or a substituted or unsubstituted alkyl, alkoxyl or alkenyl, independently substituted with one to three halogen, hydroxyl, cyano or —NR′R″ moieties;

R^{3a}, R^{4a} are absent when c is a double bond, or are each independently hydrogen, hydroxyl, halogen, alkoxyl or a substituted or unsubstituted alkyl independently substituted with one to three hydroxyl or halogen moieties when c is a single bond

R^{3b}, R^{4b}, R^{5a}, R^{6a}, R^{7a} and R^{8a} are each, independently, hydrogen, hydroxyl, halogen, alkoxyl or a substituted or unsubstituted alkyl independently substituted with one to three hydroxyl or halogen moieties, or any two of R^{6a}, R^{7a} and R^{8a} may be linked to form a 3-7 membered carbocyclic ring.

In an exemplary embodiment, the compound is represented by Formula (II), wherein R^{1a}, R^{3a} and R^{4a} are each hydrogen.

In another exemplary embodiment, the compound is represented by Formula (II), wherein c represents a single bond.

In yet another exemplary embodiment, the compound is represented by Formula (II), wherein R^{6a} and R^{8a} are both methyl.

In one embodiment, the compound is represented by Formula (II), wherein R^{1a} is hydrogen.

In another embodiment, the compound is represented by Formula (II), wherein R^{2a} is hydroxyl.

In another embodiment, the compound is represented by Formula (II), wherein R^{7a} is hydroxyl.

In yet another embodiment, the compound is represented by Formula (II), wherein R^{5a} is hydroxyl.

In certain embodiments, the vitamin D compound is 1,25-dihydroxyvitamin D3 (1,25(OH)₂D3 (also known as calcitriol); 1,25-dihydroxy-16-ene-23-yne-cholecalciferol; 1α-hydroxyvitamin D3; 1α,24-dihydroxyvitamin D3, or MC 903 (e.g., calcipotriol).

Other suitable analogs, metabolites, derivatives and/or mimics of vitamin D compounds include, for example, those described in the following patents, each of which is incorporated by reference in its entirety: U.S. Pat. Nos. 4,391,802 (1α-hydroxyvitamin D derivatives); 4,717,721 (1α-hydroxy derivatives with a 17 side chain greater in length than the cholesterol or ergosterol side chains); 4,851,401 (cyclopentano-vitamin D analogs); 4,866,048 and 5,145,846 (vitamin D3 analogues with alkynyl, alkenyl, and alkanyl side chains); 5,120,722 (trihydroxycalciferol); 5,547,947 (fluoro-cholecalciferol compounds); 5,446,035 (methyl substituted vitamin D); 5,411,949 (23-oxa-derivatives); 5,237,110 (19-nor-vitamin D compounds); 4,857,518 (hydroxylated 24-homo-vitamin D derivatives). Other suitable examples include ROCALTROL (Roche Laboratories); CALCIJEX injectable calcitriol; investigational drugs from Leo Pharmaceuticals including EB 1089 (24a,26a,27a,trihomo-22,24-diene-1α,25-(OH)-2-D3, KH 1060 (20-epi-22-oxa-24a,26a,27a-trihomola, 25-(OH)-2-D3), MC 1288 (1,25-(OH)₂-20-epi-D3) and MC 903 (calcipotriol, 1α,24s(OH)₂-22-ene-26,27-dehydro-D3); Roche Pharmaceutical drugs that include 1,25-(OH)₂-16-ene-D3, 1,25-(OH)₂-16-ene-23-yne-D3, and 25-(OH)₂-16-ene-23-yne-D3; Chugai Pharmaceuticals 22-oxacalcitriol (22-oxa-1α,25-(OH)-2-D3; 1α-(OH)-D5 from the University of Illinois; and drugs from the Institute of Medical Chemistry-Schering AG that include ZK 161422 (20-methyl-1,25-(OH)-2-D3) and ZK 157202 (20-methyl-23-ene-1,25-(OH)-2-D3); 1α-(OH)-D2; 1α-(OH)-D3, 1α-(OH)-D4, 25-(OH)-D2; 25-(OH)-D3; and 25-(OH)-D4. Additional examples include 1α,25-(OH)₂-26,27-d6-D3; 1α,25-(OH)₂-22-ene-D3; 1α,25-(OH)-2-D3; 1α,25-(OH)-2-D2; 1α,25-(OH)-2-D4; 1α,24,25-(OH)-3-D3; 1α,24,25-(OH)-3-D2; 1α,24,25-(OH)-3-D4; 1α-(OH)-25-FD3; 1α-(OH)-25-FD4; 1α-(OH)-25-FD2; 1α,24-(OH)-2-D4; 1α,24-(OH)-2-D3; 1α,24-(OH)-2-D2; 1α,24-(OH)₂-25-FD4; 1α,24-(OH)₂-25-FD3; 1α,24-(OH)₂-25-FD2; 1α,25-(OH)₂-26,27-F6-22-ene-D3; 1α,25(OH)₂-26,27-F6-D3; 1α,25S—(OH)₂-26-F3-D3; 1α,25-(OH)₂-24-F2-D3; 1α,25S,26-(OH)₂-22-ene-D3; 1α,25R,26-(OH)₂-22-ene-D3; 1α,25-(OH)-2-D2; 1α,25-(OH)₂-24-epi-D3; 1α,25-(OH)₂-23-yne-D3; 1α,25-(OH)₂-24R—F-D3; 1α,25S,26-(OH)-2-D3; 1α,24R—(OH)₂-25F-D3; 1α,25-(OH)₂-26,27-F6-23-yne-D3; 1α,25R—(OH)₂-26-F3-D3; 1α,25,28-(OH)-3-D2; 1α,25-(OH)₂-16-ene-23-yne-D3; 1α,24R,25-(OH)-3-D3; 1α,25-(OH)₂-26,27-F6-23-ene-D3; 1α,25R—(OH)₂-22-ene-26-F3-D3; 1α,25S—(OH)₂-22-ene-26-F3-D3; 1α,25R—(OH)-2-D3-26,26,26-d3; 1α,25S—(OH)-2-D3-26,26,26-d3; and 1α,25R—(OH)₂-22-ene-D3-26,26,26-d3. Additional examples can be found in U.S. Pat. No. 6,521,608, the entire disclosure of which is incorporated by reference herein. See also, e.g., U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940, 5,384,313, 5,374,629, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,804,502, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,367,177, 4,336,193, 4,360,472, 4,360,471, 4,307,231, 4,307,025, 4,358,406, 4,305,880, 4,279,826, and 4,248,791, the entire disclosures of each of which are incorporated by reference herein.

Yet other compounds which may be utilized include vitamin D mimics such as bis-aryl derivatives disclosed by U.S. Pat. No. 6,218,430 and WO publication 2005/037755, the entire disclosures of each of which are incorporated by reference herein. Additional examples of non-secosteroidal vitamin D mimic compounds suitable for the present invention can be found in U.S. Pat. Nos. 6,831,106; 6,706,725; 6,689,922; 6,548,715; 6,288,249; 6,184,422, 6,017,907, 6,858,595, and 6,358,939, the entire disclosures of each of which are incorporated by reference herein.

Yet other suitable vitamin D3 analogs, metabolites, derivatives and/or mimics which may be utilized include those identified in U.S. Patent Application Publication No. 2006/0177374, the entire disclosure of which is incorporated by reference herein.

The language “vitamin D analog” includes compounds that are similar to vitamin D in structure and function. In one embodiment, the vitamin D analog is a vitamin D3 analog (e.g., a compound that is similar to vitamin D3 in structure and function).

The language “vitamin D metabolite” includes compounds that are intermediates and the products involved in the metabolism of vitamin D. In one embodiment, the vitamin D metabolite is a vitamin D3 metabolite (e.g., a compound that is an intermediate or product involved in the metabolism of vitamin D3).

The language “vitamin D derivative” includes compound that can arise from a parent compound (e.g., vitamin D) by replacement of one atom with another atom or group of atoms. In one embodiment, the vitamin D derivative is a vitamin D3 derivative (e.g., a compound that can arise from vitamin D3 by replacement of one atom with another atom or group of atoms).

The language “vitamin D mimic” includes compounds that can chemically imitate vitamin D in a biological process. In one embodiment, the vitamin D mimic is a vitamin D3 mimic (e.g., a compound that can chemically imitate vitamin D3 in a biological process).

As used herein, the term “alkyl” includes fully saturated branched or unbranched (e.g., straight chain or linear) hydrocarbon moiety, comprising 1 to 20 carbon atoms. Preferably the alkyl comprises 1 to 7 carbon atoms, and more preferably 1 to 4 carbon atoms. Representative examples of alkyl moieties include methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl.

The term “C₁₋₇ alkyl” includes hydrocarbons having one to seven carbon atoms. Moreover, the term “alkyl” includes both “unsubstituted C₁₋₇ alkyls” and “substituted C₁₋₇ alkyls.” Representative examples of substituents for C₁₋₇ alkyl moieties are hydroxy, halogen, cyano, nitro, C₃₋₈ cycloalkyl, C₂₋₇ alkenyl, C₂₋₇ alkynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

As used herein, the term “alkoxy” includes alkyl-O—, wherein alkyl is defined herein above. Representative examples of alkoxy moieties include, but are not limited to, methoxy, ethoxy, propoxy, 2-propoxy, butoxy, tert-butoxy, pentyloxy, hexyloxy, cyclopropyloxy-, cyclohexyloxy- and the like. Preferably, alkoxy groups have about 1-7, more preferably about 1-4 carbons. The term alkoxy includes substituted alkoxy. Examples of substituted alkoxy groups include halogenated alkoxy groups. Examples of halogen substituted alkoxy groups are fluoromethoxy, difluoromethoxy, trifluoromethoxy, chloromethoxy, dichloromethoxy, and trichloromethoxy.

The term “C₁₋₇ alkoxy” includes C₁₋₇ alkyl-O—, wherein C₁₋₇ alkyl is defined above. Moreover, the term C₁₋₇ alkoxy includes both “unsubstituted C₁₋₇ alkoxy” and “substituted C₁₋₇ alkoxy.” Representative examples of substituents for C₁₋₇ alkoxy moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

The term “alkoxyalkyl” includes alkyl groups, as defined above, in which the C₁₋₇ alkyl group is substituted with C₁₋₇ alkoxy. Moreover, the term “alkoxyalkyl” includes both “unsubstituted alkoxyalkyl” and “substituted alkoxyalkyl.” Representative examples of substituents for alkoxyalkyl moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

The term “alkenyl” includes branched or unbranched hydrocarbons having at least one carbon-carbon double bond. The term “C₂₋₇ alkenyl” refers to a hydrocarbon having two to seven carbon atoms and comprising at least one carbon-carbon double bond. Representative examples of alkenyl moieties include, but are not limited to, vinyl, prop-1-enyl, allyl, butenyl, isopropenyl or isobutenyl. Moreover, the term “alkenyl” includes both “unsubstituted C₂₋₇ alkenyls” and “substituted C₂₋₇ alkenyls.”Representative examples of substituents for C₂₋₇ alkenyl moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

The term “alkynyl” includes branched or unbranched hydrocarbons having at least one carbon-carbon triple bond. The term “C₂₋₇ alkynyl” refers to a hydrocarbon having two to seven carbon atoms and comprising at least one carbon-carbon triple bond. Representative examples of C₂₋₇ alkynyl moieties include, but are not limited to, ethynyl, prop-1-ynyl (propargyl), butynyl, isopropynyl or isobutynyl. Moreover, the term “alkynyl” includes both “unsubstituted C₂₋₇ alkynyls” and “substituted C₂₋₇ alkynyls.” Representative examples of substitutents for C₂₋₇ alkynyl moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino, and C₁₋₇ alkyl C₆₋₁₀ arylamino).

As used herein, the term “cycloalkyl” includes saturated or unsaturated monocyclic, bicyclic or tricyclic hydrocarbon groups of 3-12 carbon atoms, preferably 3-8, or 3-7 carbon atoms. Exemplary monocyclic hydrocarbon groups include, for example, cyclopropyl, cyclobutyl, cyclopentyl, cyclopentenyl, cyclohexyl and cyclohexenyl. Exemplary bicyclic hydrocarbon groups include, for example, bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, and 2,6,6-trimethylbicyclo[3.1.1]heptyl, bicyclo[2.2.2]octyl. Exemplary tricyclic hydrocarbon groups include, for example, adamantyl.

The term “C₃₋₈ cycloakyl” includes cyclic hydrocarbon groups having 3 to 8 carbon atoms. Moreover, the term “C₃₋₈ cycloakyl” includes both “unsubstituted C₃₋₈ cycloakyl” and “substituted C₃₋₈ cycloakyl.” Representative examples of substitutents for C₃₋₈ cycloakyl moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

The term “aryl” includes monocyclic or bicyclic aromatic hydrocarbon groups having 6-20 carbon atoms in the ring portion. Representative examples of aryl moieties include, but are not limited to, phenyl, naphthyl, anthracyl, phenanthryl or tetrahydronaphthyl.

The term “C₆₋₁₀ aryl” includes aromatic hydrocarbon groups having 6 to 10 carbon atoms in the ring portion. Moreover, the term aryl includes both “unsubstituted aryl” and “substituted aryl.” Representative examples of substitutents for aryl moieties include, but are not limited to, hydroxy, halogen, cyano, nitro, C₁₋₇ alkyl, C₃₋₈ cycloalkyl, C₂₋₇ alkenyl, C₂₋₇ akynyl, C₁₋₇ alkoxy, C₂₋₇ alkenyloxy, C₂₋₇ alkynyloxy, halogen or amino (including C₁₋₇ alkyl amino, di-C₁₋₇ alkylamino, C₆₋₁₀ arylamino, di-C₆₋₁₀ arylamino).

The term “heteroaryl” includes monocyclic or bicyclic heteroaryl moieties, containing from 5-10 ring members selected from carbon atoms and 1 to 5 heteroatoms, selected from O, N or S. Examples of heteroaryl groups include, but are not limited to, thienyl, furyl, pyrrolyl, imidazolyl, pyrazolyl, thiazolyl, isothiazolyl, oxa-2,3-diazolyl, oxa-2,4-diazolyl, oxa-2,5-diazolyl, oxa-3,4-diazolyl, thia-2,3-diazolyl, thia-2,4-diazolyl, thia-2,5-diazolyl, thia-3,4-diazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl. A heteroaryl group may be mono-, bi-, tri-, or polycyclic.

The term “heteroaryl” further includes groups in which a heteroaromatic ring is fused to one or more aryl, cycloaliphatic, or heterocyclyl rings, where the radical or point of attachment is on the heteroaromatic ring or on the fused aryl ring. Representative examples of such heteroaryl moieties include, but are not limited to, indolyl, isoindolyl, indazolyl, indolizinyl, purinyl, quinolizinyl, quinolinyl, isoquinolinyl, cinnolinyl, phthalazinyl, naphthyridinyl, quinazolinyl, quinaxalinyl, phenanthridinyl, phenathrolinyl, phenazinyl, phenothiazinyl, phenoxazinyl, benzisoqinolinyl, thieno[2,3-b]furanyl, furo[3,2-b]-pyranyl, 5H-pyrido[2,3-d]-O— oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl, 7-benzo[b]thienyl, benzoxazolyl, benzimidazolyl, benzothiazolyl, benzoxapinyl, benzoxazinyl, 1H-pyrrolo[1,2-b][2]benzazapinyl, benzofuryl, benzothiophenyl, benzotriazolyl, pyrrolo[2,3-b]pyridinyl, pyrrolo[3,2-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-d]pyridinyl, pyrazolo[4,3-c]pyridinyl, pyrazolo[3,4-c]pyridinyl, pyrazolo[3,4-d]pyridinyl, pyrazolo[3,4-b]pyridinyl, imidazo[1,2-a]pyridinyl, pyrazolo[1,5-a]pyridinyl, pyrrolo[1,2-b]pyridazinyl, imidazo[1,2-c]pyrimidinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, or pyrimido[4,5-d]pyrimidinyl. Moreover, the term “heteroaryl” includes both “unsubstituted heteroaryl” and “substituted heteroaryl.”

The aromatic ring of an “aryl” or “heteroaryl” group can be unsubstituted or substituted at one or more ring positions with substituents including, for example, halogen, hydroxy, cyano, nitro, C₁₋₇ alkyl, C₃₋₈ cycloalkyl, C₂₋₇ alkenyl, C₂₋₇ akynyl, C₆₋₁₀ aryl, heteroaryl, heterocyclyl, C₁₋₇ alkoxy, C₃₋₈ cycloalkyloxy, C₂₋₇ alkenyloxy, C₂₋₇ alkynyloxy, C₆₋₁₀ aryloxy, heteroaryloxy, heterocyclyloxy, arylalkyloxy, heteroarylalkyloxy, heterocyclylalkyloxy, ketones (including C₁₋₇ alkylcarbonyl, C₃₋₈ cycloalkylcarbonyl, C₂₋₇ alkenylcarbonyl, C₂₋₇ alkynylcarbonyl, C₆₋₁₀ aroyl, C₆₋₁₀ aryl C₁₋₇ alkylcarbonyl, hetero arylcarbonyl, heterocyclylcarbonyl), esters (including C₁₋₇ alkoxycarbonyl, C₃₋₈ cycloalkyloxycarbonyl, C₆₋₁₀ aryloxycarbonyl, heteroaryloxycarbonyl, heterocyclyloxycarbonyl, C₁₋₇ alkylcarbonyloxy, C₃₋₈ cycloakylcarbonyloxy, C₆₋₁₀ arylcarbonyloxy, heteroarylcarbonyloxy, heterocyclylcarbonyloxy), carbonates (including C₁₋₇ alkoxycarbonyloxy, C₆₋₁₀ aryloxycarbonyloxy, heteroaryloxycarbonyloxy), carbamates (including C₁₋₇ alkoxycarboxylamino, C₆₋₁₀ aryloxycarbonylamino, C₂₋₇ alkenyloxycarbonylamino, C₂₋₇ alkynyloxycarbonylamino, C₆₋₁₀ aryloxycarbonylamino, aminocarbonyloxy, C₁₋₇ alkylaminocarbonyloxy, di-C₁₋₇ alkylaminocarbonyloxy, C₆₋₁₀ arylaminocarbonyloxy), carbamoyl (including C₁₋₇ alkylaminoacarbonyl, di-C₁₋₇ alkylaminocarbonyl, C₆₋₁₀ arylaminocarbonyl, C₆₋₁₀ aryl C₁₋₇ alkylaminocarbonyl, C₂₋₇ alkenylaminocarbonyl), amido (including C₁₋₇ alkylcarbonylamino, C₁₋₇ alkylcarbonyl C₁₋₇ alkylamino, C₆₋₁₀ arylcarbonylamino, heteroarylcarbonylamino), C₆₋₁₀ aryl C₁₋₇ alkyl, heteroaryl C₁₋₇ alkyl, heterocyclo C₁₋₇ alkyl, amino (including C₁₋₇ alkyl amino, di-C₁₋₇ alkylamino, C₆₋₁₀ arylamino, di-C₆₋₁₀ arylamino, and C₁₋₇ alkyl C₆₋₁₀ arylamino), sulfonyl (including C₁₋₇ alkylsulfonyl, C₆₋₁₀ arylsulfonyl, C₆₋₁₀ aryl C₁₋₇ alkylsulfonyl, heteroarylsulfonyl, C₁₋₇ alkoxysulfonyl, C₆₋₁₀ aryloxysulfonyl, heteroaryloxysulfonyl, C₃₋₈ cycloalkylsulfonyl, heterocyclylsulfonyl), sulfamoyl, sulfonamido, phosphate, phosphonato, phosphinato, thioether (including C₁₋₇ alkylthio, C₆₋₁₀ arylthio, heteroarylthio), ureido, imino, amidino, thiocarboxyl (including C₁₋₇ alkylthiocarbonyl, C₆₋₁₀ arylthiocarbonyl), sulfinyl (including C₁₋₇ alkylsulfinyl, C₆₋₁₀ arylsulfinyl), carboxyl, wherein each of the afore-mentioned hydrocarbon groups may be optionally substituted with one or more C₁₋₇ alkyl, C₂₋₇ alkenyl, C₂₋₇ alkynyl, C₃₋₈ cycloalkyl, halogen, hydroxy or C₁₋₇ alkoxy groups.

As used herein, the term “heterocyclyl” or “heterocyclo” includes unsubstituted or substituted, saturated or unsaturated non-aromatic ring or ring systems, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. In one embodiment, heterocyclyl moiety represents a saturated monocyclic ring containing from 5-7 ring atoms and optionally containing a further heteroatom, selected from O, S or N. The heterocyclic group can be attached at a heteroatom or a carbon atom. The heterocyclyl can include fused or bridged rings as well as spirocyclic rings. Examples of heterocyclyl moieties include, for example, dihydrofuranyl, dioxolanyl, dioxanyl, dithianyl, piperazinyl, pyrrolidine, dihydropyranyl, oxathiolanyl, dithiolane, oxathianyl, thiomorpholino, oxiranyl, aziridinyl, oxetanyl, oxepanyl, azetidinyl, tetrahydrofuranyl, tetrahydrothiophenyl, pyrrolidinyl, tetrahydropyranyl, piperidinyl, morpholino, piperazinyl, azepinyl, oxapinyl, oxaazepanyl, oxathianyl, thiepanyl, azepanyl, dioxepanyl, and diazepanyl.

The term “heterocyclyl” includes heterocyclic groups as defined herein substituted with 1, 2 or 3 substituents such as ═O, ═S, halogen, hydroxy, cyano, nitro, alkyl, cycloalkyl, alkenyl, akynyl, aryl, heteroaryl, heterocyclyl, alkoxy, cycloalkyloxy, alkenyloxy, alkynyloxy, aryloxy, heteroaryloxy, heterocyclyloxy, arylalkyloxy, heteroarylalkyloxy, heterocyclylalkyloxy, ketones (including alkylcarbonyl, cycloalkylcarbonyl, alkenylcarbonyl, alkynylcarbonyl, aroyl, arylalkylcarbonyl, heteroarylcarbonyl, heterocyclylcarbonyl), esters (including alkoxycarbonyl, cycloalkyloxycarbonyl, aryloxycarbonyl, heteroaryloxycarbonyl, heterocyclyloxycarbonyl, alkylcarbonyloxy, cycloakylcarbonyloxy, arylcarbonyloxy, heteroarylcarbonyloxy, heterocyclylcarbonyloxy), carbonates (including alkoxycarbonyloxy, aryloxycarbonyloxy, heteroaryloxycarbonyloxy), carbamates (including alkoxycarboxylamino, aryloxycarbonylamino, alkenyloxycarbonylamino, alkynyloxycarbonylamino, aryloxycarbonylamino, aminocarbonyloxy, alkylaminocarbonyloxy, dialkylaminocarbonyloxy, arylaminocarbonyloxy), carbamoyl (including alkylaminoacarbonyl, dialkylaminocarbonyl, arylaminocarbonyl, arylakylaminocarbonyl, alkenylaminocarbonyl), amido (including alkylcarbonylamino, alkylcarbonylalkylamino, arylcarbonylamino, heteroarylcarbonylamino), arylalkyl, heteroarylalkyl, heterocyclylalkyl, amino (including alkyl amino, dialkylamino, arylamino, diarylamino, and alkylarylamino), sulfonyl (including alkylsulfonyl, arylsulfonyl, arylalkylsulfonyl, heteroarylsulfonyl, alkoxysulfonyl, aryloxysulfonyl, heteroaryloxysulfonyl, cycloakylsulfonyl, heterocyclylsulfonyl), sulfamoyl, sulfonamido, phosphate, phosphonato, phosphinato, thioether (including alkylthio, arylthio, heteroarylthio), ureido, imino, amidino, thiocarboxyl (including alkylthiocarbonyl, arylthiocarbonyl), sulfinyl (including alkylsulfinyl, arylsulfinyl), carboxyl wherein each of the afore-mentioned hydrocarbon groups may be optionally substituted with one or more C₁₋₇ alkyl, C₂₋₇ alkenyl, C₂₋₇ alkynyl, C₃₋₈ cycloalkyl, halogen, hydroxy or C₁₋₇ alkoxy groups.

The term “heterocyclylalkyl” is an C₁₋₇ alkyl substituted with heterocyclyl. The term includes unsubstituted and substituted heterocyclylalkyl moieties which may be substituted with one or more C₁₋₇ alkyl, C₂₋₇ alkenyl, C₂₋₇ alkynyl, C₃₋₈ cycloalkyl, halogen, hydroxy or C₁₋₇ alkoxy groups.

The term “carbonyl” or “carboxy” includes compounds and moieties which contain a carbon connected with a double bond to an oxygen atom (C═O). The carbonyl can be further substituted with any moiety which allows the compounds of the invention to perform its intended function. For example, carbonyl moieties may be substituted with C₁₋₇ alkyls, C₂₋₇ alkenyls, C₂₋₇ alkynyls, C₆₋₁₀ aryls, C₁₋₇ alkoxy, aminos, etc. Examples of moieties which contain a carbonyl include aldehydes, ketones, carboxylic acids, amides, esters, urea, anhydrides, etc.

The term “hydroxy” or “hydroxyl” includes groups with an —OH or —O⁻.

The term “halogen” includes fluorine, bromine, chlorine, iodine, etc.

The term “perhalogenated” includes moieties in which all hydrogens are replaced by halogen atoms.

The vitamin D compounds of the invention, or their pharmaceutically acceptable salts, solvates or prodrugs thereof, may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

The language “stereoisomer” includes compounds made up of the same atoms bonded by the same bonds but having different three-dimensional structures, which are not interchangeable. The present invention contemplates various stereoisomers and mixtures thereof and includes “enantiomers,” which refers to two stereoisomers whose molecules are nonsuperimposeable minor images of one another.

The present invention includes all pharmaceutically acceptable isotopically-labeled vitamin D compounds in which one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Isotopically-labeled vitamin D compounds can generally be prepared by conventional techniques known to those skilled in the art or by processes analogous to those described in the accompanying Examples and Preparations Sections using an appropriate isotopically-labeled reagent in place of the non-labeled reagent previously employed.

One of the exemplary vitamin D compounds of the invention is 1,25(OH)₂D3, which is mainly synthesized by the proximal tubules of the kidneys, from a number of precursors. Another secondary source of 1,25(OH)₂D3 is through the conversion of less active metabolites by the skin in response to sunlight. 1,25(OH)₂D3 is a secosteroid which has been shown to regulate calcium influx and efflux into cells as well as mobilizing calcium to the skeleton. In addition, 1,25(OH)₂D3 has other cellular roles irrespective of calcium regulation, mainly by interacting with vitamin D receptor (VDR). The VDR is a nuclear receptor; however, it can also be found in the cytoplasmic region. The consensus is that the VDR, a steroidal receptor, located in the nucleus, interacts with other receptors such as the retinoid X receptor.

While the effects of 1,25(OH)₂D3 are incompletely understood, it is known that it also exerts a non-calcemic role and has genomic effects due to its affinity to the DNA-binding domain of VDR. The DNA-binding domain of VDR regulates protein-protein interaction as well as other co-factors, and the activation of the functional domain. The ligand-binding domain (LBD) is vital for phosphorylation, an important factor in the transcriptional activity of VDR.

The low molecular weight and lipophilic properties of 1,25(OH)₂D3 ensure its entry into the cell membrane, and its high affinity towards the VDR leads to its binding the ligand-binding domain of the VDR. 1,25(OH)₂D3 indirectly recruits histone acetylases, thereby opening chromatin. Consequently, co-activator target genes are switched on by co-activators. On the other hand, without LBD binding, VDR can also lead to the repression of transcription mediated by the histone deacetylases by interacting with other repressor proteins. Gene transcription is mediated by the VDR response elements, which are specific DNA sequences in the promoter regions of the genes.

In addition to the genomic actions, 1,25(OH)₂D3 also regulates influx and efflux of calcium and chloride. 1,25(OH)₂D3 further regulates mitogen-activated protein kinases (MAP-kinases), leading to rapid proliferative inhibition and cellular differentiation.

The term “prodrug” includes compounds that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood or conversion in the gut or liver. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, Anglican Pharmaceutical Association arid Pergamon Press, 1987.

The language “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The language “pharmaceutically acceptable salt” includes both acid and base addition salts.

The language “pharmaceutically acceptable acid addition salt” includes those salts which retain the biological effectiveness and properties of the free bases, which are not biologically or otherwise undesirable, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphorirc acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

The language “pharmaceutically acceptable base addition salt” includes those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention (e.g., a vitamin D compound). As used herein, the term “solvate” includes an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

The language “pharmaceutical composition” includes formulations of a compound of the invention (e.g., a vitamin D compound) and a medium generally accepted in the art, for delivery of the biologically active compound of the invention to a subject. Such a medium includes all pharmaceutically acceptable carriers, diluents or excipients thereof.

Pharmaceutical compositions comprising the vitamin D compound and/or the chemotherapeutic agent of the present invention may be administered to the subject orally, systemically, parenterally, topically, rectally, nasally, intravaginally or intracisternally. They are, of course, given by forms suitable for each administration route. For example, they are administered in tablets or capsule form, by injection, inhalation, ointment, etc., administration by injection, infusion or inhalation; topical by lotion or ointment; and rectal or vaginal suppositories.

The phrases “parenteral administration” and “administered parenterally” as used herein include modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion administration.

The phrases “systemic administration,” “administered systemically,” as used herein, includes the administration of the vitamin D compounds other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” include an area of tissue that covers external surfaces of a body, or which lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, foams, pastes, ointments, salves, balms, douches or drops.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Standard composition strategies for topical agents can be applied to the vitamin D compounds in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate that is solid at room temperature, but liquid at body temperature and, therefore, will melt in the rectum or vaginal cavity and release the active agent. Compositions which are suitable for vaginal administration also include pessaries, tampons, creams, gels, pastes, foams, films, or spray compositions containing such carriers as are known in the art to be appropriate. The carrier employed in the pharmaceutical compositions of the invention should be compatible with vaginal administration.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolatum. Exemplary ophthalmic compositions include eye ointments, powders, solutions and the like.

Powders and sprays can contain, in addition to the vitamin D compounds, carriers such as lactose, talc, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the vitamin D compounds together with conventional pharmaceutically acceptable carriers and stabilizers. The carriers and stabilizers vary with the requirements of the particular compound, but typically include nonionic surfactants (e.g., Tweens, Pluronics, polyethylene glycol and the like), proteins like serum albumin, sorbitan esters, oleic acid, lecithin, amino acids such as glycine, buffers, salts, sugars or sugar alcohols. Aerosols generally are prepared from isotonic solutions. Generation of the aerosol or any other means of delivery of the present invention may be accomplished by any of the methods known in the art. For example, in the case of aerosol delivery, the compound is supplied in a finely divided form along with any suitable carrier with a propellant.

Liquefied propellants are typically gases at ambient conditions and are condensed under pressure. The propellant may be any acceptable and known in the art including propane and butane, or other lower alkanes, such as those of up to 5 carbons. The composition is held within a container with an appropriate propellant and valve, and maintained at elevated pressure until released by action of the valve.

The vitamin D compounds can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A vitamin D compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the vitamin D compound only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients. Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the vitamin D compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the vitamin D compounds can be aqueous or oleaginous suspension. These suspensions may be formulated according to techniques known in the art using suitable dispersing or wetting agents and suspending agents.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. The sterile injectable preparation may also be a sterile injectable solution or suspension in a nontoxic parenterally-acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant. The vitamin D compounds will represent some percentage of the total dose in other dosage forms in a material forming a combination product, including liquid solutions or suspensions, suppositories, douches, enemas, gels, creams, emulsions, lotions, slurries, powders, sprays, foams, pastes, ointments, salves, balms, douches, drops and others.

In one embodiment, the vitamin D compound may be administered prophylactically. For prophylactic applications, the vitamin D compound can be applied prior to potential CIM. The timing of application can be optimized to maximize the prophylactic effectiveness of the vitamin D compound. The timing of application will vary depending on the mode of administration, doses, the stability and effectiveness of composition, the frequency of the dosage, e.g., single application or multiple dosage. One skilled in the art will be able to determine the most appropriate time interval required to maximize prophylactic effectiveness of the vitamin D compound.

The vitamin D compound when present in a composition will generally be present in an amount from about 0.000001% to about 100%, more preferably from about 0.001% to about 50%, and most preferably from about 0.01% to about 25% of total weight.

For compositions of the present invention comprising a carrier, the composition comprises, for example, from about 1% to about 99%, preferably from about 50% to about 99%, and most preferably from about 75% to about 99% by weight of at least one carrier.

Also, the separate components of the compositions of the invention may be preblended or each component may be added separately to the same environment according to a predetermined dosage for the purpose of achieving the desired concentration level of the treatment components and so long as the components eventually come into intimate admixture with each other. Further, the present invention may be administered or delivered on a continuous or intermittent basis.

In some embodiments, wherein the vitamin D compound is formulated as a sterile solution comprising between about 50 μg/mL and about 400 μg/mL, for example, between about 100 μg/mL and 350 μg/mL, between about 150 μg/mL and about 300 μg/mL or between about 200 μg/mL and about 250 μg/mL of the vitamin D compound. In yet other embodiments, the vitamin D compound is formulated as a sterile solution comprising between about 50 μg/mL and about 100 μg/mL, for example, between about 55 μg/mL and about 95 μg/mL, between about 60 μg/mL and about 90 μg/mL, between about 65 μg/mL and about 80 μg/mL, and between about 70 μg/mL and about 75 μg/mL of the vitamin D compound. In still other embodiments, the vitamin D compound is formulated as a sterile solution comprising between about 300 μg/mL and about 400 μg/mL, for example, between about 310 μg/mL and about 380 μg/mL, between about 330 μg/mL and about 370 μg/mL or between about 340 μg/mL and between about 350 μg/mL and of vitamin D compound. In one embodiments, comprises about 75 μg/mL vitamin D compound. In another embodiment, the formulation comprises about 345 μg/mL vitamin D compound. In a further embodiment, vitamin D compound is calcitriol.

In other embodiments, the formulation further comprises anhydrous undenatured ethanol and polysorbate 20. In yet another embodiments, the formulation is diluted 1:10 in 0.9% sodium chloride solution prior to administration to the subject.

In some embodiments, the vitamin D compound is prepared as a sterile calcitriol formulation of between about 50 μg/mL and about 400 μg/mL in a vehicle of anhydrous 200 proof (U.S.) undenatured ethanol, USP (96% w/w) and polysorbate 20, USP (4% w/w), and diluted 1:10 in 0.9% sodium chloride solution (USP) prior to administration to the host.

In certain embodiments, the vitamin D compound is prepared as a sterile calcitriol formulation at 75 μg/mL or 345 μg/mL, in a vehicle of anhydrous 200 proof (U.S.) undenatured ethanol, preferably USP grade or better (96% w/w) and polysorbate 20, preferably USP grade or better (4% w/w), and diluted 1:10 in 0.9% sodium chloride solution (USP grade or better) prior to administration to the host.

In accordance with the present disclosure, a vitamin D compound, such as vitamin D3, or analogs, metabolites, derivatives and/or mimics thereof, may be administered in conjunction with chemotherapeutic agents, to reduce undesirable side effects of these chemotherapeutic agents, including CIM. The vitamin D compounds may be administered prior to, simultaneously with, or subsequently to the administration of the chemotherapeutic agent to provide the desired effect.

While not wishing to be bound by any particular theory, the methods of the present invention may ameliorate myelosuppression by increasing the availability of pluripotent stem cell progenitors. Such methods can be used in combination with standard therapy (e.g., those employing granulocyte-stimulating factor or G-CSF) to increase proliferation of myeloid cells and/or improve their mobilization from the bone marrow, thereby diminishing the dose and administration of colony-stimulating factors (CSFs) as well as the recuperation time following chemotherapy.

The vitamin D compounds of the invention may modulate bone marrow progenitors and stromal cells prior to the administration of antineoplastic agents. The methods herein may be used in combination with standard therapy (e.g., those using G-CSF) to increase proliferation of myeloid cells and/or improve their mobilization from the bone marrow, thereby diminishing the dose and administration of colony-stimulating factors (CSFs) as well as the recuperation time following chemotherapy.

Another aspect of the invention provides methods to determine the optimal dosage of the subject vitamin D compounds (such as vitamin D3), including derivatives, analogs and/or active metabolites thereof, that may be administered to a patient. In certain embodiments, the vitamin D compounds of the invention may be administered to myeloid cells of a host (sometimes referred to herein, in certain embodiments, as a patient) to determine an optimal therapeutic dose. Preferably, the optimal therapeutic dose protects the myeloid cells without eliciting a hypercalcemic effect.

Methods which may be utilized to detect viability of the myeloid cells are known in the art, including (without limitation), manual and automated trypan blue exclusion, dye exclusion, methods using fluorometric exclusion dyes, immunofluorescent and direct microscopy, the use of radioactive isotopes and scintillation to determine cellular function or viability, the use of colony assays such as semi-solid agar colony formation assays or methylcellulose assays, methods to detect early markers of apoptosis using different substrates, such as caspases, and any other automated or manual method by which one can determine whether a specific dose of the subject vitamin D compound is cytotoxic.

It should be noted that all embodiments described herein (above and below) are contemplated to be able to combine with any other embodiment(s) where applicable, including embodiments described only under one of the aspects of the invention, and embodiments described under different aspects of the invention.

### EXAMPLES

The following Examples are being submitted to illustrate embodiments of the present invention. These Examples are intended to be illustrative only, and are not intended to limit the scope of the invention in any respect. Parts and percentages are by weight unless otherwise indicated. As used herein, “room temperature” refers to a temperature of from about 20° C. to about 25° C.

### Example 1

### Chemo-Protective Effect of 1,25(OH)₂D3

### Materials and Methods

1,25(OH)₂D3, human recombinant GM-CSF and G-CSF, histopaque 1077 were purchased from Sigma-Aldrich (St. Louis, Mo.). 4-hydroxyperoxycylophosphamide

(4HC), the active metabolite of the chemotherapeutic drug cyclophosphamide, was obtained from Duke Comprehensive Cancer Center. Tissue culture grade agar, fetal calf serum (FCS), and powdered Dulbecco's Modified Eagle's Medium (DMEM) were obtained from Invitrogen (Carlsbad, Calif.). Peripheral circulating progenitor stem cells were obtained by venipuncture of the saphenous vein of a healthy male donor into sodium heparin vacutainers (Becton, Dickinson and Company, Franklyn Lakes N.J.). The buffy coat was obtained by gradient centrifugation using histopaque 1077 as per manufacturer's instructions. Cells were washed twice with RMPI 1640 supplemented with 10% fetal calf serum (Invitrogen).

A colony formation assay including semi-solid medium formulated with DMEM and 0.5% agar was used. For these cultures, mononuclear cells were plated at a concentration of about 2.5×10⁵ cells/mL, and GM-CSF and G-CSF were added at a concentration of about 100 U/mL. Cells were cultured for 14 days in a 5% CO₂ incubator, with 100% humidity at 37° C.

At the end of the culture period, colonies (clusters of 50 or more cells) were counted using an inverted microscope by two independent viewers.

### Results

Peripheral stem cells were randomized into 4 groups at a concentration of 5×10⁵ cells/mL in DMEM supplemented with 10% fetal calf serum. Group 1 was an untreated control, group 2 was incubated for 24 hours with 0.05 μg/mL of 1,25(OH)₂D3, group 3 was incubated for 24 hours with 0.05 μg/mL of 1,25(OH)₂D3, group 4 was untreated.

Cells were washed with DMEM 10% fetal calf serum. Groups 3 and 4 were then incubated with 25 μg/mL of 4-HC for 20 hours. Subsequently, all groups were washed twice as previously described. Cells were then plated in semi-solid agar medium as described above.

Results of the 4 groups described are shown in Table 1 below. The results confirm the chemo-protective effect of 1,25(OH)₂D3.

TABLE 1
Colony Counts at 14 Days

| Group 1 | Group 2 | Group 3 | Group 4 |
|---|---|---|---|
| Untreated | 1,25(OH)₂D3 | 1,25(OH)₂D3 + 4- | 4-HC |
| control | | HC | |
| 50 ± 7 | 48 ± 6 | 37 ± 5 | 0 ± 0 |

* Results are means of experiments conducted in quadruplicates; ±Standard Deviation)

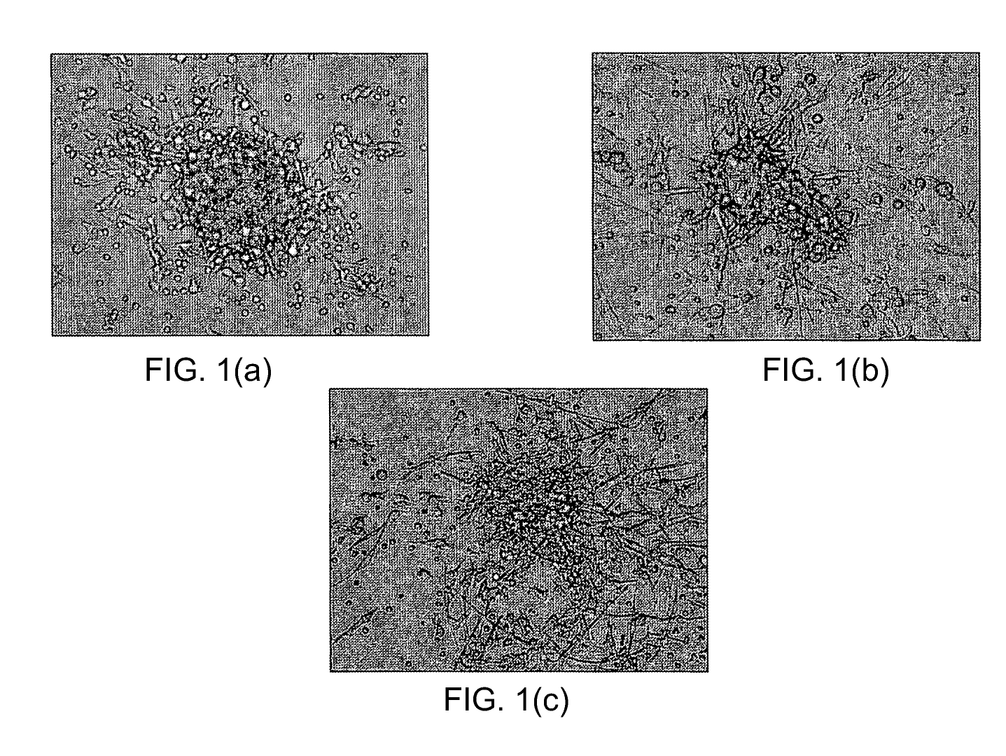

Photomicrographs of the myeloid colonies were also obtained and are provided in FIGS. 1A, 1B and 1C. FIG. 1A shows a normal myeloid colony derived from peripheral blood supplemented with growth factors. As can be seen in FIG. 1B, with 1,25(OH)₂D3 at the protective dose, myeloid colonies were also observed. In addition, colonies were observed in plates in which 1,25(OH)₂D3 protected from 4-HC-induced toxicity (FIG. 1C), while no colonies were observed in plates with 4-HC alone. This demonstrates that 1,25(OH)₂D3 at a dose of 0.05 μg/mL for 24 hours protects myeloid progenitors against the effect of toxicants such as 4-HC.

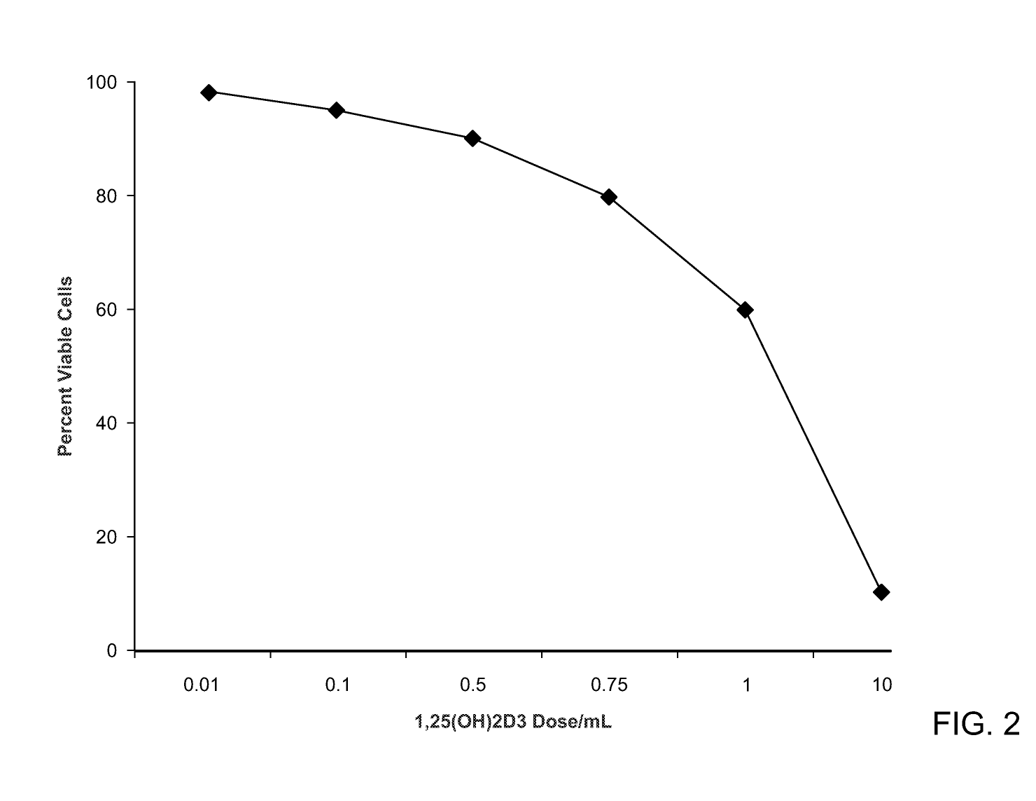

Varying doses of 1,25(OH)₂D3 were applied to the myeloid cells. A graph of the effects of 1,25(OH)₂D3 on myeloid cells is provided as FIG. 2. The viability of the myeloid cells was determined by trypan blue exclusion after a 24-hour exposure to varying doses of 1,25(OH)₂D3. For these experiments, 2.5×10⁵ cells/mL were incubated with different doses of 1,25(OH)₂D3 (0.01 μg/mL, 0.1 μg/mL, 0.5 μg/mL, 0.75 μg/mL, 1 μg/mL, and 10 μg/mL) for 24 hours in RPMI 1640 supplemented with 10% fetal calf serum. As can be seen in FIG. 2, at the optimal protective dose of 0.05 μg/mL, viability was 90%.

### Example 2

### High Dose Non-Calcemic Regimen (NCR) of API 31543 (Calcitriol) for the Treatment of Chemotherapy-Induced Myelosuppression (CIMS)

The objective of this study is to evaluate the potential protective effect against CIMS of the test article, Compound 31543 (Calcitriol, USP), using an animal model of multi-course CIMS bearing MIAC51, a rat chloroleukemia cell line developed by gastric instillation of 20-methylcolanthrene and subsequent injection of the chloroleukemic cells into rat neonates. The resulting cell line is a malignant myelogenous leukemia with features of human chloroleukemia (leukemia, leukemic ascites and chloroma formation).

Two separate sterile calcitriol concentrates are used in this study. Specifically, sterile calcitriol concentrates of 75 μg/mL and 345 μg/mL are prepared in a vehicle of anhydrous 200 proof undenatured ethanol, USP (96% w/w) and Polysorbate 20, USP (4% w/w). The concentrates are diluted 1:10 at time of use with Sodium Chloride (0.9%) Solution for Injection, USP. For example, an aliquot of 1.0 mL of the 75 μg/mL Calcitriol concentrate mixed with 4.0 mL of Sodium Chloride Solution for Injection will give a 15 μg/mL calcitriol aliquot solution. Injection of 0.17 mL of the aliquot would deliver approximately 2.6 μg of calcitriol. A 1.0 mL aliquot of the 345 μg/mL concentrate mixed with 4.0 mL of Sodium Chloride (0.9%) Solution for Injection will give approximately 69 μg/mL calcitriol aliquot solution. Injection of 0.15 mL of this aliquot would deliver 10.4 μg of calcitriol. The lower concentration is used for the young rats (21-day old) and the higher calcitriol concentration is used to dose the older rats (49-day old).

The vehicle control is the vehicle concentrate of sterile anhydrous 200 proof undenatured ethanol, USP (96% w/w) and Polysorbate 20, USP (4% w/w) diluted with Sodium Chloride (0.9%) Solution for Injection, USP at an equivalent dilution ratio (1 mL concentrate vehicle+4 mL isotonic saline). Final dosing concentration is determined in advance via a preliminary dosing study in the animal model.

Sprague Dawley rats (10-day old rat pups, preferably of natural litters) are used in this study. In a study conducted by Peter et al., administration of vinblastine to male Lewis rats led to a sharp decrease in total leukocyte count and absolute neutrophil count (ANC) (Peter et al., 1998). In addition, Peter et al. have demonstrated that rats are an excellent counterpart to the human with respect to granulocyte-colony stimulating factor (G-CSF). Thus, in rats the onset of neutropenia as judged by the nadir of ANC has been well characterized. Moreover, the rat model also has the advantage of being responsive to frequently used myelosuppresive chemotherapies such as cyclophosphamide, doxorubicin and paclitaxel and combinations thereof (Jimenez and Yunis, 1992). The neonatal rat model of leukemia, developed by Dr. Jimenez, is the only rat chloroleukemia model in the world and provides an optimal opportunity to simultaneously test any effect of the test compound on the development of CIM, the treatment of leukemia, potential interaction with chemotherapeutic agents, and the effect of the test agent on prevention of CIM.

Rats are kept in litters of 10 up to day 21 of age. On day 21, rats are separated and housed in pairs with a unique identifier number assigned. For these experiments there are two tiers:

Stage 1: Pups 14-day old to 32-day old rats. MIAC51 cells are injected on day 15. A first pulse of vehicle or API 31543 is administered on day 21, and 3 different chemotherapy regimens starting on day 22 and ending on day 24 are then given. The nadir of total leukocyte count is observed between days 4-6 after administration of chemotherapy, while it is between days 2 to 7 for NCA (Peter et al., 1998). Post-mortem bone marrow cultures and calcium measurements are performed on days 22 and 26. A final blood count and bone marrow culture in a percentage of animals are performed on day 32. Animals with overt leukemia are sacrificed.

Stage 2: 47- to 60-day old rats. On day 47, rats with advanced leukemia are sacrificed. On day 48, a second pulse of test article or vehicle is administered. Bone marrow cultures and plasma calcium level analysis are performed on day 49 to assess the effect of the test article on the bone marrow. Chemotherapy is started and continued until day 52. On day 54, a second culture of bone marrow cells and calcium levels are tested. Finally, animals are sacrificed on day 60 after a complete blood count.

Table 2 outlines the study design:

TABLE 2

| | | | | |
|---|---|---|---|---|
| | Number | | | |
| | of Pups | Treatment | | |
| Group | per | (Vehicle or | Chemotherapy | Number of |
| Number | Group | Calcitriol) | Regimen | Pups |

Stage 1: 14- to 32-Day Old Pups

| | | | | |
|---|---|---|---|---|
| I | 60 | Vehicle | Cyclophosphamide | 120 total |
| | 60 | Calcitriol | | |
| II | 60 | Vehicle | Cyclophosphamide | 120 total |
| | 60 | Calcitriol | Doxorubicin | |
| III | 60 | Vehicle | Cyclophosphamide | 120 total |
| | 60 | Calcitriol | Doxorubicin | |
| | | | Paclitaxel | |

Stage 2: 47- to 60-Day Old Pups

| | | | | |
|---|---|---|---|---|
| I | 10 | Vehicle | Cyclophosphamide | 20 total |
| | 10 | Calcitriol | | |
| II | 40 | Vehicle | Cyclophosphamide | 80 total |
| | 40 | Calcitriol | Doxorubicin | |
| III | 40 | Vehicle | Cyclophosphamide | 80 total |
| | 40 | Calcitriol | Doxorubicin | |
| | | | Paclitaxel | |

Test article and vehicle are administered intravenously, and chemotherapies are injected intraperitoneally.

The dose of calcitriol used in pulse therapy for myelodysplasia is 45 μg. Using the Mosteller calculation, for an average person of 5′8″, with an ideal weight of 151 lbs, body surface area (BSA) is 1.81 m² (Halls, 2008). Thus, the dose is 25 μg/m² for humans (Whitehouse and Curd, 2007). To calculate BSA, the Meeh-Rubner calculation Ab=km^{2/3} is used. The skin surface area (SSA) can be estimated with almost absolute precision (r=>0.9) (Spiers and Candas, 1984).

For a 21-day old rat, SSA is 102 cm², while for a 49-day old rat, SSA is 399 cm². Thus, the initial calcitriol pulse dose that is tested is approximately 2.6 μg for the 21-day old rat and approximately 10 μg for the 49-day old. A range of doses, for example between 0.26 μg and 2.6 μg for the 21-day old rat and between 1 μg and 10 μg for the 49-day old, is tested to determine whether this dose is accurate or should be increased or decreased.

The test article and vehicle are administered on the day prior to chemotherapy both in the first and second cycle. The test article will be dosed as indicated above, e.g., at either 2.6 μg or 10 μg, in the first and second cycles. Chemotherapies are given based on weight in a volume of approximately 100 μL intraperitoneally. Table 3, below, provides the chemotherapy doses and schedules.

TABLE 3
Chemotherapy Regimen

| | Dose | Schedule |
|---|---|---|
| Cyclophosphamide | 150 mg/kg | 1 × 1 day |
| Cyclophosphamide | 100 mg/kg | 1 × 1 day |
| Doxorubicin | 25 mg/kg | 1 × 3 days |
| Cyclophosphamide | 100 mg/kg | 1 × 1 day |
| Doxorubicin | 25 mg/kg | 1 × 3 days |
| Paclitaxel | 10 mg/kg | 1 × 3 days |

Animals are monitored daily for lethargy, anorexia or other signs of distress in response to chemotherapy. All animals showing signs of premature leukemia such as leukemic ascites are summarily sacrificed and recorded.

To inject MIAC51 cells, fifteen days old rats are manually restrained, and their right legs are gently pulled. The area to be injected is cleaned with an alcohol swab. Then 1×10⁵ MIAC51 cells are injected intraperitoneally.

To administer test and control article in the first calcitriol pulse, each litter of rats are administered either vehicle or test article intravenously through the tail vein in a volume of 100-200 μL.

To administer test and control article in the second calcitriol pulse, survivors that have been demonstrated to be cancer-free according to the hematological analysis are anesthetized with a ketamine/xylazine cocktail (50 mg/kg and 5 mg/kg, respectively) on day 48, and the test compound or control article is injected intravenously through the tail vein for a second time.

To administer the first chemotherapy course in the 22-day old rats, which receive either a chemotherapy regime, chemotherapy regime and test article, or chemotherapy regime and vehicle (e.g., as described in Table 3 above), an average weight of each litter is obtained and used to prepare a suitable concentration of chemotherapy. Chemotherapies are then injected intraperitoneally in a volume of approximately 100 μL according to the individual weight of the animals using 29 ga. ½ cc insulin syringes. At this age, no anesthesia is necessary. The right legs are gently pulled and the area to be injected is cleaned with an alcohol swab.

To administer a second chemotherapy course in the 49-day old rats, which either receive a chemotherapy regime, chemotherapy regime and test article, or chemotherapy regime and vehicle (e.g., as described in Table 3 above), an average weight of the rats is obtained and used to prepare a suitable concentration of chemotherapy. Animals are then anesthetized with a ketamine/xylazine prior to injection of antineoplastic agents. Chemotherapies will be injected intraperitoneally in a volume of approximately 100 μL according to the individual weight of the animals using 29 ga. ½ cc insulin syringes.

Chemotherapies used in the experiments are prepared in a chemical hood, and are transferred to 50 mL conical polypropylene tubes and tightly capped. Paclitaxel is dissolved at a concentration of 50 mg/mL in DMSO, and is aliquoted and stored at −20° C. prior to use. To improve the solubility of cyclophosphamide in distilled water, 750 mg of D-mannitol/1 g of cyclophosphamide is added. Doxorubicin is fully soluble in distilled water.

The tubes containing the chemotherapies in powder are tightly capped and transferred to the biosafety cabinet in which they are diluted using distilled water according to the preferred dosage predetermined for the weight of the animals (approximately 100 μL/rat). The container with either the water soluble chemotherapies and/or D-mannitol is then filtered to sterility using a 0.2 μm low protein binding membrane filter and a syringe into a sterile conical polypropylene tube. The sterile stock solutions of etoposide and paclitaxel can be mixed with the other chemotherapies in distilled water after they are filtered in polypropylene tubes according to the average weight of the rats. Chemotherapies are transferred into individual 29 ga. ½ cc syringes (Becton Dickinson and Company) under sterile conditions.

MIAC51 cells are cultured in a 5% CO₂ incubator with 100% humidity at 37° C. as previously described (Jimenez and Yunis, 1987, incorporated by reference). Cells are grown in non-tissue culture-treated flasks (Falcon) in RPMI 1640 medium (Gibco Invitrogen, Carlsbad, Calif.) supplemented with L-glutamine and 10% fetal bovine serum (Gibco Invitrogen, Carlsbad, Calif.). Prior to the injection of cells into the animals, they are grown to 50% confluency and collected in conical tubes. Cells are then centrifuged at 600 g for 10 minutes at room temperature, and resuspended at a concentration 1×10⁶ in RPMI 1640 without fetal bovine serum. The cell suspension is then transferred to 29 gauge (ga). ½ cc insulin syringes under sterile conditions.

To assay the Colony-Forming Activity of bone marrow progenitors and MIAC51 cells, bone marrow cells are obtained as previously described (Jimenez and Yunis, 1988), and are washed with serum free DMEM. Cells are then suspended to a concentration of 1×10⁶/mL and layered onto a gradient for centrifugation for 40 minutes at 400 g. The pellet found between the medium and gradient is then carefully aspirated and washed in serum free DMEM two times. Finally, a cell suspension containing 1×10⁵ cells/mL is prepared in DMEM supplemented with 10% fetal bovine serum, and incubated in tissue culture plates for 3 hours. The non-adherent cells are aspirated and transferred to semi-solid agar culture plates.

To prepare semi-solid agar medium, powdered MEM is reconstituted in tissue-culture grade water to a concentration of 2×. Agar (0.3%) is then added and the mixture is boiled until the agar is fully dissolved (Perkins and Yunis, 1986). The medium is cooled to 37° C. and essential amino acids which might have been depleted during the boiling process are then added. The semi-solid medium is then distributed onto multi-well clusters, filling one well with tissue culture grade water to avoid further evaporation. At this point, G- or GM-CSF are added following manufacturer's procedure, and the bone marrow cell suspension or MIAC51 cells are added by careful pipetting in order to avoid bubbles. Colonies are counted 7 days later.

To prepare semi-solid agar stained slides, plates after 7 days are fixed with a dilution of 30% acetic acid in ethanol for 30 minutes, followed by absolute ethanol, 30% ethanol, and 50% ethanol at 3-minute intervals. Thereafter, the contents of the plates are transferred onto a 3 inch by 2 inch glass slide and stained with Harris' Alum hematoxylin. Colonies are scored as previously described (Jimenez and Yunis, 1988).

To conduct hematological analysis, blood smears are done throughout both courses of chemotherapy, starting one day prior to the pulse of calcitriol and ending 10 days later. Animals are anesthetized using a cocktail of ketamine 50 mg/kg/xylazine 5 mg/kg. The tail vein is cleaned with an alcohol swab and punctured using a sterile 29 ga. Syringe, and 50 μL blood is obtained to make a blood smear. For blood counts, a small volume of blood is obtained and used to count cells in a blood counter. The presence of myeloid cells and MIAC51 in peripheral blood smears are evaluated by routine stain of slides using Wright's stain.

On days 22, 26, 49 and 53, blood from 3 animals is collected by cardiac puncture. All blood samples are collected in a vial for analysis of calcium levels. All animals used for bone marrow cultures are anesthetized and exsanguinated prior to obtaining the bone marrow.

To collect femoral bone marrow, animals are exsanguinated as described above. Using a size 20 scalpel, an incision is made in the inguinal area, and the muscles are cut. Using sterile forceps, the bone is debrided until the epiphiseal surface is readily seen. Femurs are then separated from their joints using a sterile bone cutter. Both ends of the bone are cut, and a 5 mL syringe equipped with an 18 gauge needle is used to pass RPMI 1640 supplemented with 10% Fetal Bovine Serum through the femur. The bone remaining marrow suspension is then enriched by gradient centrifugation using histopaque 1077. After 2 washes with medium, a rich mononuclear cell preparation is obtained. To make bone marrow smears, the suspension is fixed onto slides using a cytocentrifuge (Shandon, N.Y.). The actual count is calculating by accounting for the dilution factor of the medium wash. The presence of myeloid cells and MIAC51 in bone marrow smears are evaluated by routine stain of slides using Wright's stain.

Both the test article as well as the vehicle itself are tested. Each group consists of 60 animals, which is statistically significant for this study. All animals are injected with MIAC51 when they are 15 days of age.

The most myelosuppressive regimes are used for this study, including 3 chemotherapy regimens: cyclophosphamide, cyclophosphamide and doxorubicin, as well as cyclophosphamide, doxorubicin and paclitaxel. All groups receive MIAC51. The groups are: chemotherapy alone, chemotherapy+vehicle, chemotherapy+test article (a total of 180 animals per chemotherapy regimen). The final number of animals used are: 3 combination chemotherapy regimens×180 animals=540 rats.

To obtain a power of 0.8 and a=0.05 with an absolute difference of 20%, 36 animals per group may be needed. Remission rate with cyclophosphamide is at least 20%. According to power analysis, the minimum sample size to achieve statistical significance is 36 animals. Therefore, 4 more animals are added to each group to account for model attrition rate of 10%.

The analysis of the joint effect of the chemotherapies and the protective compound is performed using a two-way analysis of variance, with specific attention to the interaction between the compounds, chemotherapy and development of CIMS. A significant interaction indicates either synergy or antagonism between the two. The analysis of variance is followed by a pair wise-comparison of the differences between the responses to the protective compound in the presence or absence of chemotherapy or leukemia. Finally, development of leukemia is compared using the Fischer's Exact Probability Test. All comparisons are made at alpha=0.05.

### Example 3

### High Dose Non-Calcemic Regimen of Calcitriol for the Treatment of Chemotherapy-Induced Myelosuppression: a Study Using the Multi-Chemotherapy Regimens Model (MC<R) of Chloroleukemic Rats

For the first cycle of experiments, 15-day old Long Evans rats were injected with MIAC51. On day 21, the rats were randomized into 3 groups for each chemotherapy regimen in which Group I received vehicle and Group II received 10 μg calcitriol. A pulse dose of vehicle or calcitriol was given four days prior to chemotherapy administration. Groups I and II were each separated on day 21 into 3 groups that received the following chemotherapeutic regimens: cyclophosphamide (150 mg/kg), cyclophosphamide and doxorubicin (100 mg/kg, 25 mg/kg, respectively) and cyclophosphamide, doxorubicin and paclitaxel (100 mg/kg, 25 mg/kg, 10 mg/kg, respectively). Starting on day 20 through 32, complete granulocyte counts were then performed by puncturing the tail vein with a 27 gauge syringe while animals were manually restrained.

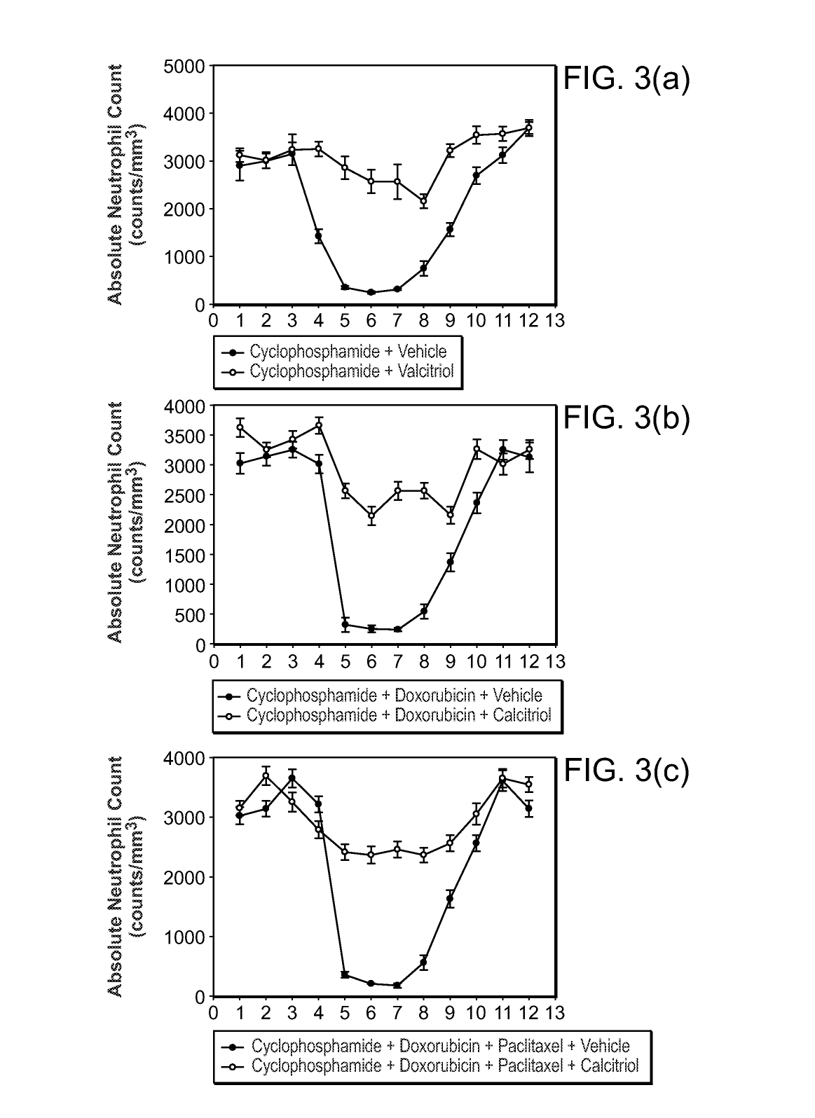

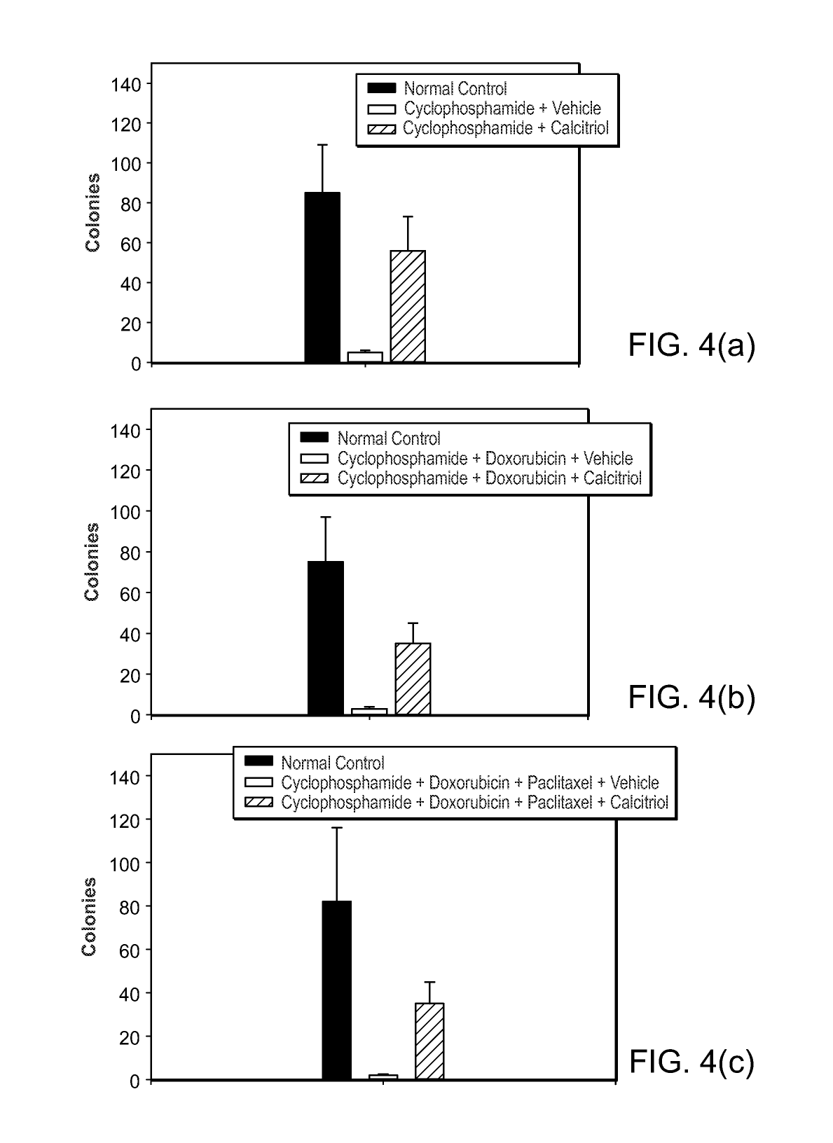

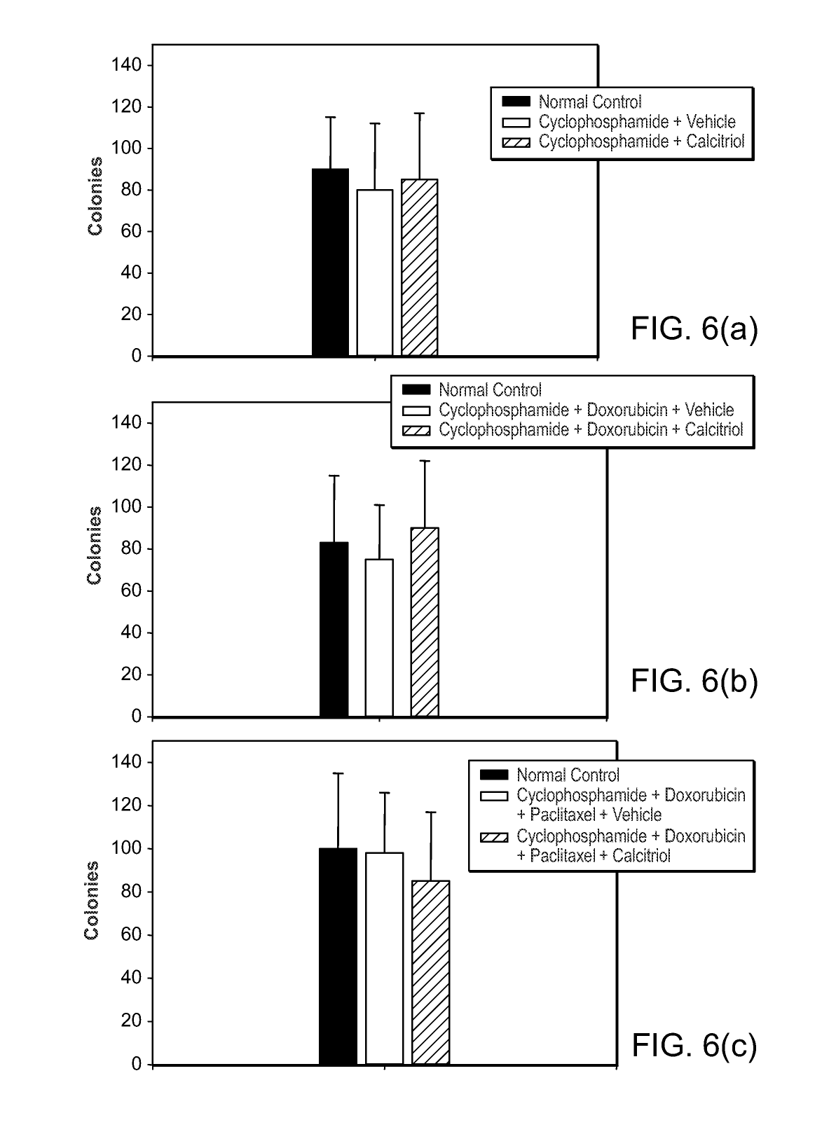

As shown in FIGS. 3(a) to 3(c), baseline absolute neutrophil counts (ANC) prior to chemotherapy administration ranged from 3621±154 mm³ to 3000±254 mm³ Once chemotherapy was administered, ANC values dropped significantly between days 24 and 27, as shown in FIGS. 4-6 and in Table 4, below.

TABLE 4

| | Nadir ANC | |
|---|---|---|
| | without calcitriol | Nadir ANC with |
| Treatment Regimen | treatment | calcitriol treatment |
| Cyclophosphamide | 245 ± 25/mm³ | 2154 ± 147/mm³ |
| Cyclophosphamide | 200 ± 25/mm³ | 2365 ± 145/mm³ |
| and doxorubicin | | |
| Cyclophosphamide, | 180 ± 38/mm³ | 2365 ± 125/mm³ |
| doxorubicin and | | |
| paclitaxel | | |

These results demonstrate that administration of calcitriol significantly decreases the nadir ANC upon administration of all three chemotherapy regimens.

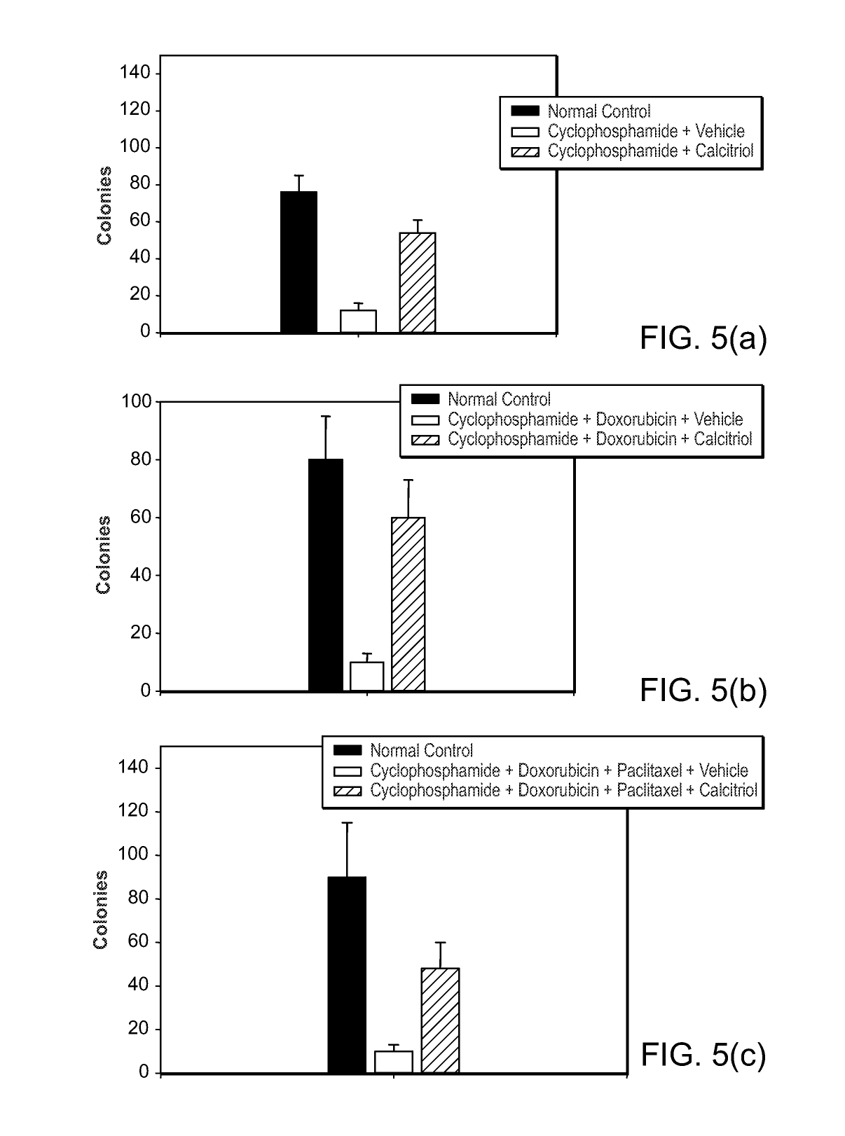

Bone marrow cultures were performed on days 22, 25 and 32. On day 32, a complete leukocyte count was performed in all animals and those who were positive for MIAC51 were sacrificed. Bone marrow cultures supported the ANC data, as illustrated in FIGS. 4-6. For the cyclophosphamide regimen, the control on day 22 was 85±24 colonies, for the group receiving cyclophosphamide and vehicle, the colony count was 5±1 colonies, and for the group receiving cyclophosphamide and calcitriol, the colony count was 56±17 colonies (FIG. 4a). On day 25, control values were 76±9 colonies, while cyclophosphamide and vehicle-treated rat bone marrow cultures were 12±4 colonies. Administration of calcitriol resulted in a significant increase in colony counts, to 80±15 colonies (FIG. 5a). Similar results can be observed with the other two chemotherapy regimens (FIGS. 4(b), 4(c), 5(b) and 5(c)).

For the second cycle of chemotherapy, survivors were re-randomized and were treated with the same regimens. Neutrophil counts were measured by puncturing the tail vein as described above. The second pulse of calcitriol was administered on day 48, and chemotherapy was started at the doses mentioned above. On day 52, rats were randomized into 3 groups for each chemotherapy regimen. Within each chemotherapy regimen, Group I received vehicle only, Group II received 20 μg calcitriol.

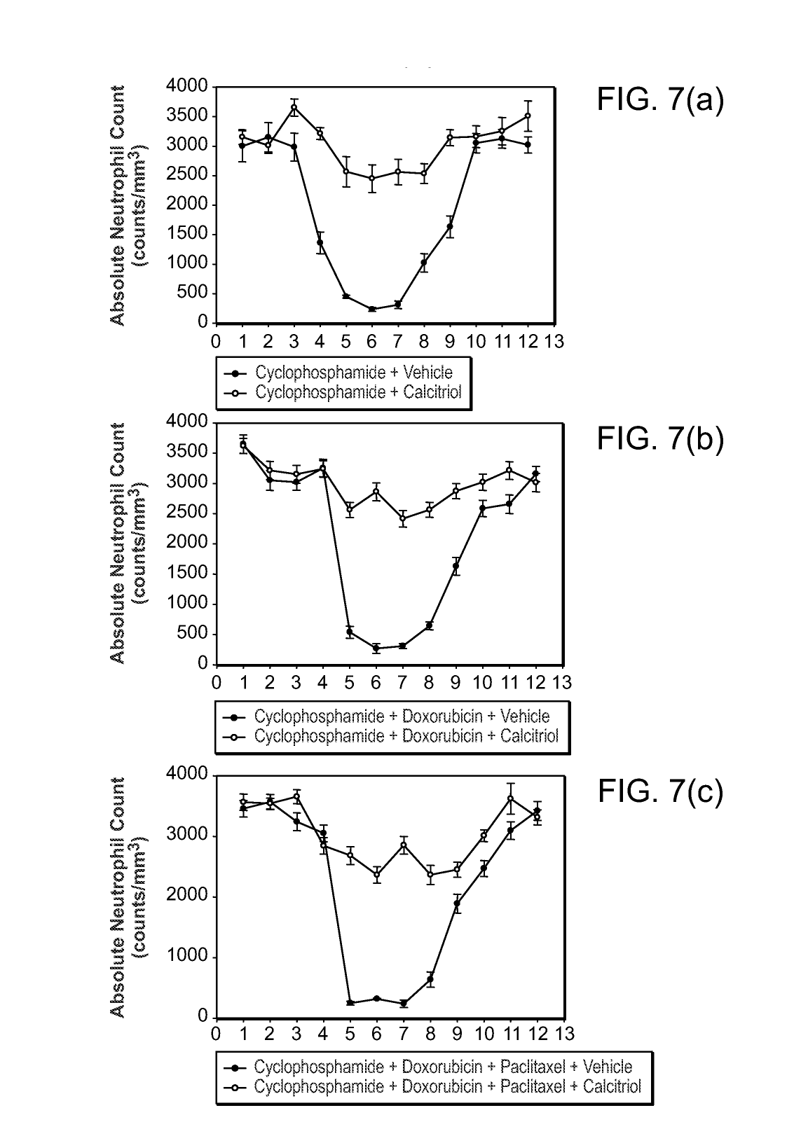

On days 32 to 60, baseline ANC prior to chemotherapy administration ranged from 3330±135 mm³ to 3005±142 mm³. As observed in the first cycle described above, upon administration of chemotherapy during the second cycle, ANC values dropped significantly between days 36 and 39, as illustrated in FIG. 7 and Table 5, below.

TABLE 5

| | Nadir ANC | |
|---|---|---|
| | without calcitriol | Nadir ANC with |
| Treatment Regimen | treatment | calcitriol treatment |
| Cyclophosphamide | 236 ± 32/mm³ | 2451 ± 235/mm³ |
| Cyclophosphamide | 27 ± 8/mm³ | 2417 ± 136/mm³ |
| and doxorubicin | | |
| Cyclophosphamide, | 240 ± 6/mm³ | 2364 ± 136/mm³ |
| doxorubicin and | | |
| paclitaxel | | |

These results demonstrate that administration of calcitriol significantly protects against chemotherapy-induced neutropenia in all three chemotherapy regimens

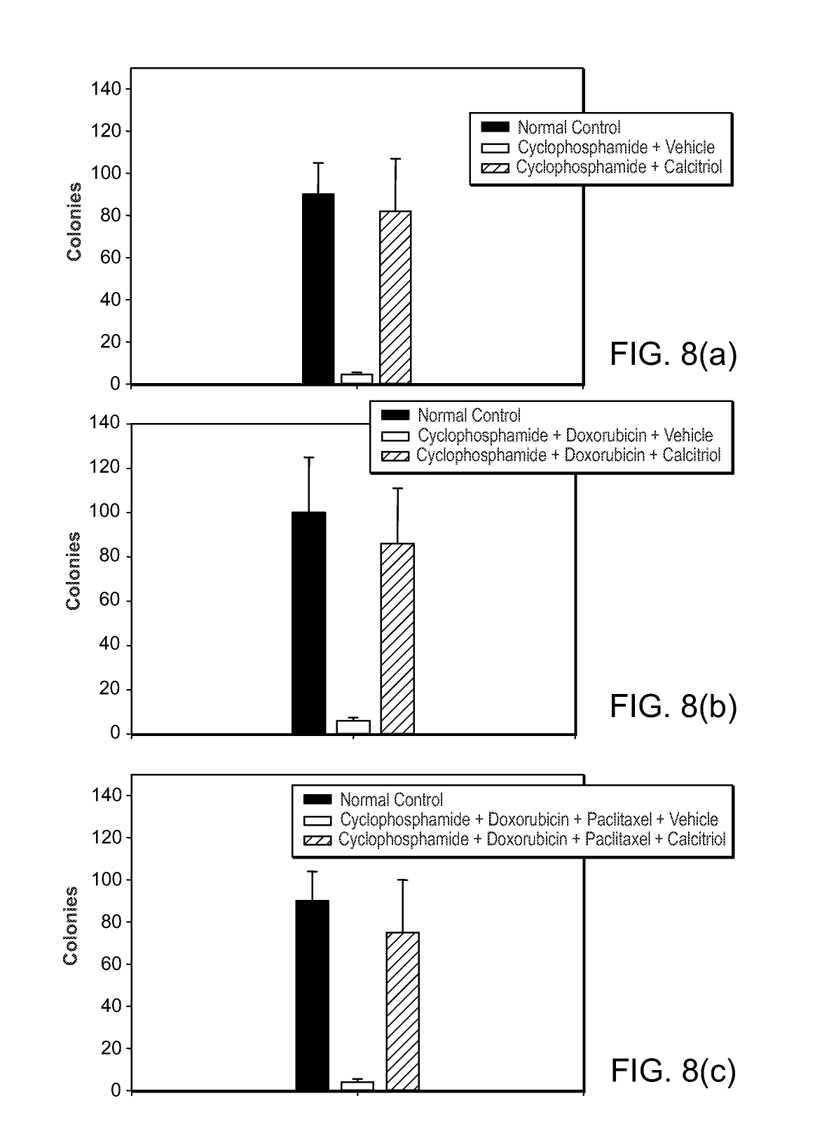

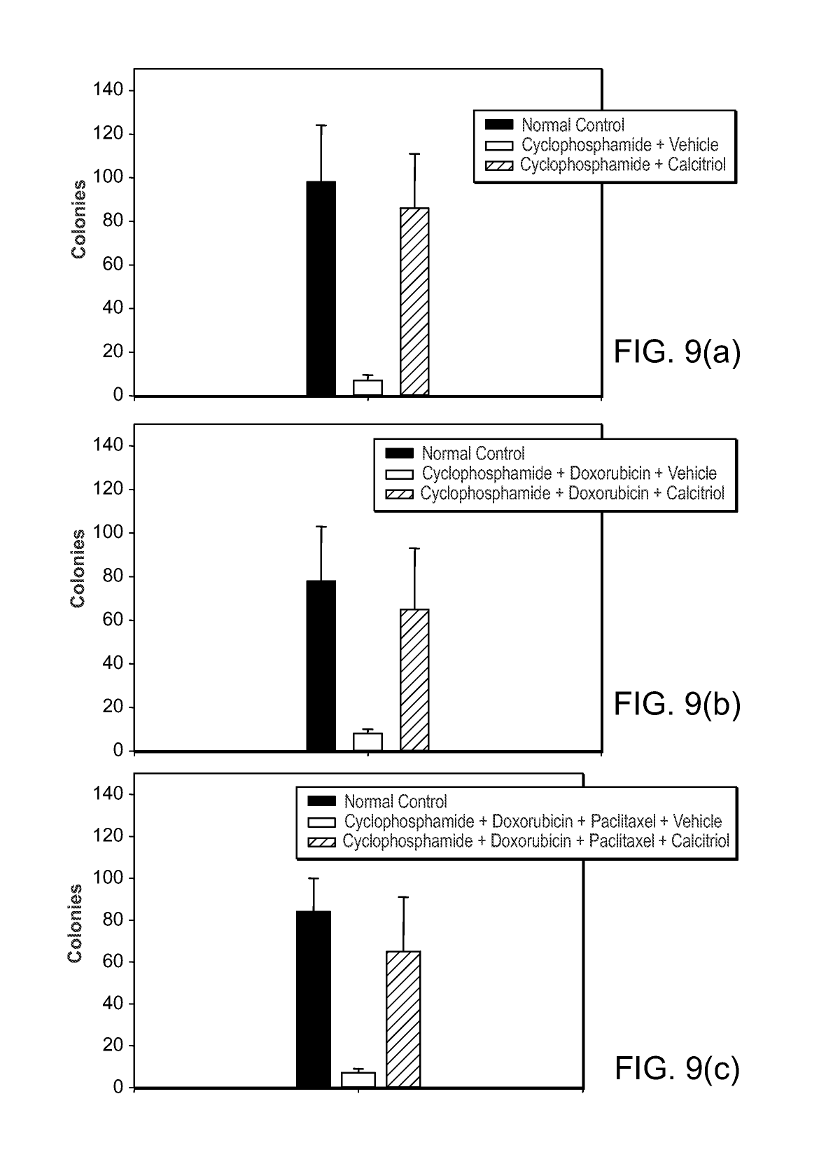

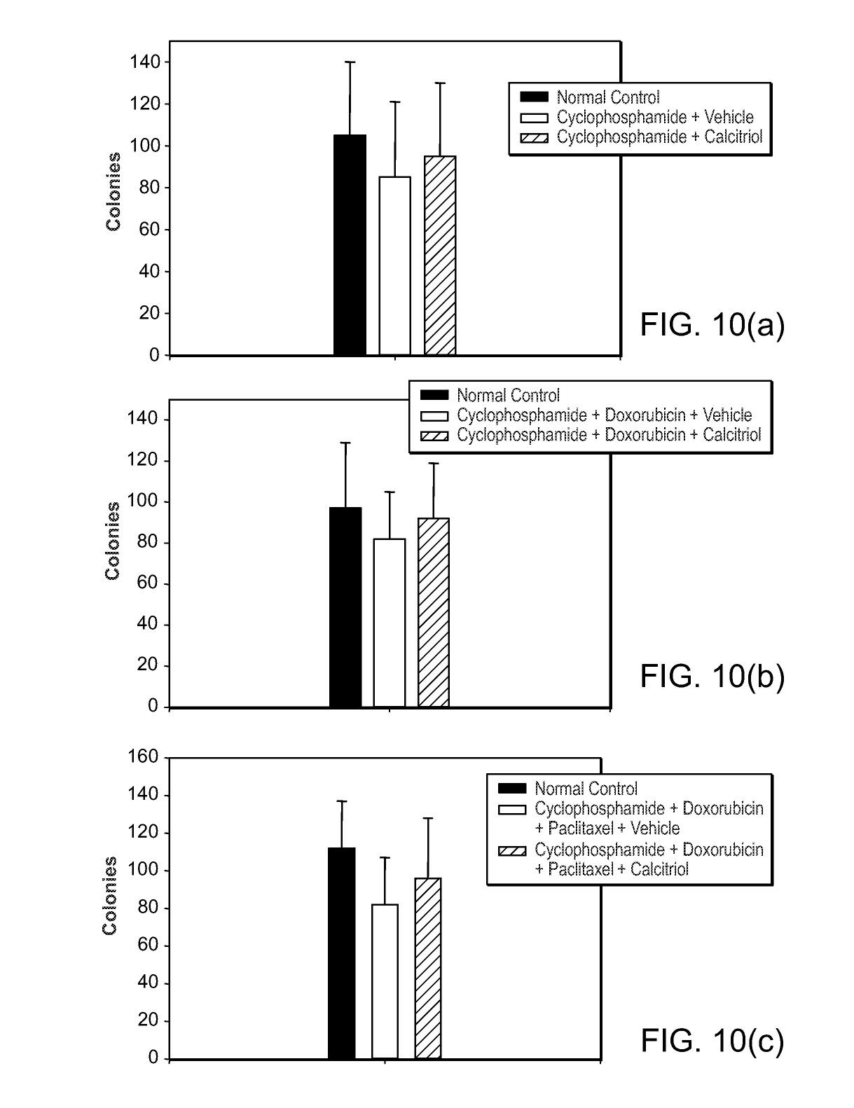

Bone marrow cultures were performed on days 49, 52 and 60 (data shown in FIGS. 8-10). Once again, the bone marrow cultures supported the ANC data. For the cyclophosphamide regimen, at day 40, the control was 90±15 colonies, the group receiving cyclophosphamide and vehicle the colony count was 4.5±1 colonies, and for the group receiving cyclophosphamide and calcitriol the colony count to 82±25 colonies. On day 52, control values were 98±26 colonies, while cyclophosphamide-treated rat bone marrow cultures were 7±2.5 colonies. Administration of calcitriol resulted in a significant increase in colony counts, with 86±25 colonies. Similar results were observed with the other two chemotherapy regimens.

Calcium levels were also measured on days 22, 25, 32, 49, 52 and 60 and the results are summarized in Table 6. In the case of cyclophosphamide, control calcium levels ranged from 10.05±day 22, 10±0.5 on day 25 and 10.5±0.3 on day 32. In rats receiving cyclophosphamide, a single pulse of calcitriol did not induce hypercalcemia. Similar results observed with the other two chemotherapeutic regimens.

TABLE 6
Calcium Levels (mg/dL)

| | | Day | Day | Day | Day | Day | |
|---|---|---|---|---|---|---|---|
| | | 22 | 25 | 32 | 49 | 52 | Day 60 |
| cyclophos- | Control | 10 ± 0.5 | 10 ± 0.5 | 10 ± 0.3 | 10.2 ± 0.3 | 11 ± 0.4 | 10 ± 0.3 |
| phamide | Chemo + | 11 ± 0.3 | 9.5 ± 0.2 | 9.5 ± 0.5 | 11 ± 0.2 | 10 ± 0.5 | 11 ± 0.4 |
| | Vehicle | | | | | | |
| | Chemo + | 10.5 ± 0.4 | 10.2 ± 0.3 | 10.5 ± 0.7 | 10 ± 0.4 | 9 ± 0.3 | 9.5 ± 0.3 |
| | calcitriol | | | | | | |
| cyclophos- | Control | 10.8 ± 0.2 | 11.2 ± 0.36 | 9.8 ± 2.3 | 9 ± 0.2 | 11 ± 0.2 | 10 ± 0.4 |
| phamide + | Chemo + | 10.25 ± 0.3 | 9.8 ± 0.5 | 11 ± 3.5 | 9.5 ± 0.1 | 9.5 ± 0.3 | 11.5 ± 0.4 |
| doxorubicin | Vehicle | | | | | | |
| | Chemo + | 10 ± 0.5 | 10 ± 0.5 | 9 ± 0.4 | 10.2 ± 0.3 | 11 ± 0.4 | 10 ± 0.3 |
| | calcitriol | | | | | | |
| Cyclophos- | Control | 11 ± 0.3 | 9.5 ± 0.2 | 10 ± 0.6 | 11 ± 0.2 | 10 ± 0.5 | 11 ± 0.4 |
| phamide + | Chemo + | 10.5 ± 0.4 | 10.2 ± 0.3 | 11.4 ± 0.2 | 10 ± 0.4 | 9 ± 0.3 | 9.5 ± 0.3 |
| doxorubicin + | Vehicle | | | | | | |
| paclitaxel | Chemo + | 10.8 ± 0.2 | 11.2 ± 0.36 | 10 ± 0.2 | 9 ± 0.2 | 11 ± 0.2 | 10 ± 0.4 |
| | calcitriol | | | | | | |

### REFERENCES

Biesma, B., E. Vellenga, et al. (1992). “Effects of hematopoietic growth factors on chemotherapy-induced myelosuppression.” Crit. Rev Oncol Hematol 13(2): 107-34.
Bociek, R. G. and J. O. Armitage (1996). “Hematopoietic growth factors.” CA Cancer J Clin 46(3): 165-84.
Freedman, L. P. (1999). “Transcriptional targets of the vitamin D3 receptor-mediating cell cycle arrest and differentiation.” J Nutr 129(2S Suppl): 581S-586S.
Halls, S. (2008). “Body Surface Area BSA Calculator Medication Doses; www.halls.md/body-surface-area/bsa.htm.” Retrieved Jul. 6, 2009, 2009.
Jimenez, J. J. and A. A. Yunis (1987). “Tumor-Cell Rejection through Terminal Cell-Differentiation.” Science 238(4831): 1278-1280.
Jimenez, J. J. and A. A. Yunis (1988). “Treatment with monocyte-derived partially purified GM-CSF but not G-CSF aborts the development of transplanted chloroleukemia in rats.” Blood 72(3): 1077-80.
Jimenez, J. J. and A. A. Yunis (1992). “Protection from chemotherapy-induced alopecia by 1,25-dihydroxyvitamin D3.” Cancer Res 52(18): 5123-5.
Katschinski, D. M., G. J. Wiedemann, et al. (1999). “Whole body hyperthermia cytokine induction: a review, and unifying hypothesis for myeloprotection in the setting of cytotoxic therapy.” Cytokine Growth Factor Rev 10(2): 93-7.
Marangolo, M., C. Bengala, et al. (2006). “Dose and outcome: the hurdle of neutropenia (Review).” Oncol Rep 16(2): 233-48.
Middleton, M. and N. Thatcher (1998). “G- and GM-CSF.” Int J Antimicrob Agents 10(2): 91-3.
Moeenrezakhanlou, A., L. Shephard, et al. (2008). “Myeloid cell differentiation in response to calcitriol for expression CD11b and CD14 is regulated by myeloid zinc finger-1 protein downstream of phosphatidylinositol 3-kinase.” J Leukoc Biol 84(2): 519-28.
Moreb, J., J. R. Zucali, et al. (1989). “Protective effects of IL-1 on human hematopoietic progenitor cells treated in vitro with 4-hydroperoxycyclophosphamide.” J Immunol 142(6): 1937-42.
Mughal, T. I. (2004). “Current and future use of hematopoietic growth factors in cancer medicine.” Hematol Oncol 22(3): 121-34.
Perkins, S. L. and A. A. Yunis (1986). “Pattern of colony-stimulating activity in HL-60 cells after phorbol-ester-induced differentiation.” Exp Hematol 14(5): 401-5.
Peter, F. W., D. A. Schuschke, et al. (1998). “Leukocyte behavior in a free-flap model following chemotherapy and application of granulocyte colony-stimulating factor (GCSF).” Microsurgery 18(4): 290-7.
Spiers, D. E. and V. Candas (1984). “Relationship of skin surface area to body mass in the immature rat: a reexamination.” J Appl Physiol 56(1): 240-3.
Sredni, B., M. Albeck, et al. (1995). “Bone marrow-sparing and prevention of alopecia by AS101 in non-small-cell lung cancer patients treated with carboplatin and etoposide.” J Clin Oncol 13(9): 2342-53.
Whitehouse, M. and J. Curd (2007). Methods of using vitamin D compounds in the treatment of myelodysplastic syndromes. United States of America.
Yunis, A. A., J. J. Jimenez, et al. (1984). “Further evidence supporting an in vivo role for colony-stimulating factor.” Exp Hematol 12(11): 838-43.

It will be appreciated that various of the above-disclosed and other features and functions, or alternatives thereof, may be desirably combined into many other different systems or applications. In addition, in view of the invention described herein, various alternatives, modifications, variations or improvements not explicitly described may be subsequently implemented by those skilled in the art. Such alternatives, modifications, variations or improvements are also intended to be encompassed by the following claims.

What is claimed is:

1. A method of preventing or reducing chemotherapy-induced myelosuppression in a subject being treated with a chemotherapeutic agent which induces myelosuppression, comprising administering to the subject an effective amount of a vitamin D compound or a pharmaceutically acceptable salt, prodrug or solvate thereof.

2. The method of claim 1, wherein the vitamin D compound is of Formula (I): 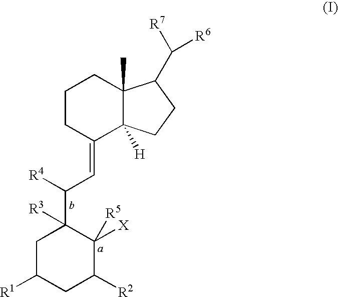
wherein
a and b are each independently a single or double bond
X is —CH₂ when a is a double bond, or X is hydrogen or a hydroxyl substituted alkyl when a is a single bond;
R¹ is hydrogen, hydroxyl, alkoxy, tri-alkyl silyl or a substituted or unsubstituted alkyl, independently substituted with one to three halogen, hydroxyl, cyano or —NR′R″ moieties;
R² is hydrogen, hydroxyl, —O-trialkyl silyl, or a substituted or unsubstituted alkyl, alkoxyl or alkenyl, independently substituted with one to three halogen, hydroxyl, cyano or —NR′R″ moieties;
R³ is absent when b is a double bond or R³ is hydrogen, hydroxyl or alkyl, or R³ and R¹ together with the carbon atoms to which they are attached may be linked to form 5-7 membered carbocyclic ring when b is a single bond;
R⁴ is hydrogen, halogen or hydroxyl;
R⁵ is absent when a is a double bond or R⁵ is hydrogen, halogen or hydroxyl when a is a single bond;
R⁶ is a substituted or unsubstituted alkyl, alkenyl, alkynyl, cycloalkyl, heterocyclicyl, alkyl-O-alkyl, alkyl-CO₂-alkyl independently substituted with one to five, hydroxyl, oxo, halogen, alkoxyl, aryl, heteroaryl, cyano, nitro or —NR′R″ moieties;
R⁷ is a substituted or unsubstituted alkyl independently substituted with one to three hydroxyl, halogen, alkoxyl, aryl, heteroaryl, cyano, nitro or —NR′R″ moieties; and,
R′ and R″ are each, independently, hydrogen, hydroxyl, halogen, —C₁₋₇ alkyl or —C₁₋₇ alkoxyl such that said CIM is prevented or reduced.

3. The method of claim 1, wherein the vitamin D compound is represented by Formula (II): 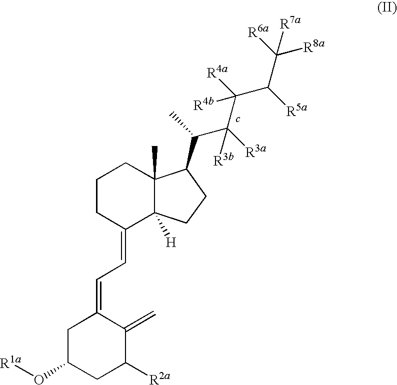
wherein
c is a single or double bond;
R^{1a} is hydrogen, tri-alkyl silyl or a substituted or unsubstituted alkyl, independently substituted with one to three halogen, hydroxyl, cyano or —NR′R″ moieties;
R^{2a} is hydrogen, hydroxyl, —O-trialkyl silyl, or a substituted or unsubstituted alkyl, alkoxyl or alkenyl, independently substituted with one to three halogen, hydroxyl, cyano or —NR′R″ moieties;
R^{3a}, R^{4a} are absent when c is a double bond, or are each independently hydrogen, hydroxyl, halogen, alkoxyl or a substituted or unsubstituted alkyl independently substituted with one to three hydroxyl or halogen moieties when c is a single bond
R^{3b}, R^{4b}, R^{5a}, R^{6a}, R^{7a} and R^{8a} are each, independently, hydrogen, hydroxyl, halogen, alkoxyl or a substituted or unsubstituted alkyl independently substituted with one to three hydroxyl or halogen moieties, or any two of R^{6a}, R^{7a} and R^{8a} may be linked to form a 3-7 membered carbocyclic ring.

4. The method of claim 1, wherein said vitamin D compound comprises 1,25-dihydroxyvitamin D3; 1,25-dihydroxy-16-ene-23-yne-cholecalciferol; 1,25-dihydroxy-16-ene-yne-cholecalciferol; 1α-hydroxyvitamin D3; 1α,24-dihydroxyvitamin D3, MC 903, or combinations thereof.

5. The method of claim 1, wherein said vitamin D compound is administered topically or systemically.

6. The method of claim 1, wherein the chemotherapy involves the use of a cell cycle-specific chemotherapeutic agent.

7. The method of claim 1, wherein the chemotherapy involves the use of a nonspecific cell cycle chemotherapeutic agent.

8. The method of claim 1, wherein the chemotherapeutic agent is a cell cycle-specific agent in combination with a nonspecific cell cycle agent.

9. The method of claim 1, wherein said vitamin D compound is administered prior to the administration of said chemotherapeutic agent.

10. The method of claim 1, wherein said vitamin D compound is co-administered with said chemotherapeutic agent.

11. The method of claim 1, wherein the subject is a mammal.

12. The method of claim 1, wherein the vitamin D compound is co-administered with an additional agent that counteracts chemotherapy-induced anemia.

13. The method of claim 12, wherein the agent is a growth factor.

14. The method of claim 13, wherein said growth factor is G-CSF or EPO.

15. The method of claim 1, wherein the vitamin D compound is formulated as a sterile solution comprising between about 50 μg/mL and about 400 μg/mL of the vitamin D compound.

16. The method of claim 15, wherein the formulation further comprises anhydrous undenatured ethanol and polysorbate 20.

17. The method of claim 15, wherein the formulation is diluted 1:10 in 0.9% sodium chloride solution prior to administration to the subject.

18. The method of claim 15, wherein the formulation comprises about 75 μg/mL vitamin D compound.

19. The method of claim 15, wherein the formulation comprises about 345 μg/mL vitamin D compound.

20. The method of claim 15, wherein the vitamin D compound is calcitriol.

21. A method to determine an optimal therapeutic dose of a vitamin D compound, comprising
administering to a subject a series of test amounts of the vitamin D compound or a pharmaceutically acceptable salt thereof, and
determining the minimal dose required to protect the myeloid cells of the subject from chemotherapy-induced myelosuppression without eliciting a hypercalcemic effect, wherein the vitamin D compound is represented by Formula (I): 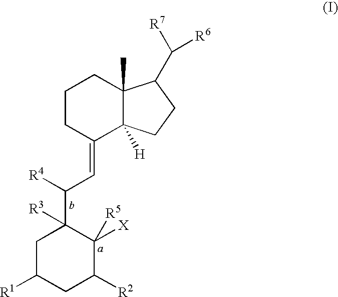
wherein
a and b are each independently a single or double bond
X is —CH₂ when a is a double bond, or X is hydrogen or a hydroxyl substituted alkyl when a is a single bond;
R¹ is hydrogen, hydroxyl, alkoxy, tri-alkyl silyl or a substituted or unsubstituted alkyl, independently substituted with one to three halogen, hydroxyl, cyano or —NR′R″ moieties;
R² is hydrogen, hydroxyl, —O-trialkyl silyl, or a substituted or unsubstituted alkyl, alkoxyl or alkenyl, independently substituted with one to three halogen, hydroxyl, cyano or —NR′R″ moieties;
R³ is absent when b is a double bond or R³ is hydrogen, hydroxyl or alkyl, or R³ and R¹ together with the carbon atoms to which they are attached may be linked to form 5-7 membered carbocyclic ring when b is a single bond;
R⁴ is hydrogen, halogen or hydroxyl;
R⁵ is absent when a is a double bond or R⁵ is hydrogen, halogen or hydroxyl when a is a single bond;
R⁶ is a substituted or unsubstituted alkyl, alkenyl, alkynyl, cycloalkyl, heterocyclicyl, alkyl-O-alkyl, alkyl-CO₂-alkyl independently substituted with one to five, hydroxyl, oxo, halogen, alkoxyl, aryl, heteroaryl, cyano, nitro or —NR′R″ moieties;
R⁷ is a substituted or unsubstituted alkyl independently substituted with one to three hydroxyl, halogen, alkoxyl, aryl, heteroaryl, cyano, nitro or —NR′R″ moieties; and, R′ and R″ are each, independently, hydrogen, hydroxyl, halogen, —C₁₋₇ alkyl or —C₁₋₇ alkoxyl .

22. A method of reducing the risk of or preventing a myelosuppression-induced disorder in a subject being treated with a chemotherapeutic agent that induces myelosuppression, comprising administering to the subject an effective amount of a vitamin D compound or a pharmaceutically acceptable salt, prodrug or solvate thereof such that said myelosuppression-induced disorder is prevented of the risk of myelosuppression-induced disorder is reduced.

23. The method of claim 22, wherein said myelosuppression-induced disorder is myelosuppression-induced infection.

24. A methods of preventing depletion of neutrophils in a subject being treated with a chemotherapeutic agent comprising, administering to the subject an effective amount of a vitamin D compound or a pharmaceutically acceptable salt, prodrug or solvate thereof, such that the depletion of neutrophils in said subject is prevented.
